(19) United States
(12) Patent Application Publication
(10) Pub. No.: US 2022/0387552 A1
(43) Pub. Date: Dec. 8, 2022

(54) METHODS OF MODULATING HAIR FOLLICLE STEM CELL QUIESCENCE BY MODULATING DERMAL NICHE ACTIVATOR GAS6

(71) Applicant: President and Fellows of Harvard College, Cambridge, MA (US)

(72) Inventors: Ya-Chieh Hsu, Cambridge, MA (US); Sekyu Choi, Cambridge, MA (US)

(21) Appl. No.: 17/774,140

(22) PCT Filed: Nov. 9, 2020
(86) PCT No.: PCT/US2020/059745

§ 371 Date: May 3, 2022

Related U.S. Application Data
(60) Provisional application No. 62/932,501, filed on Nov. 7, 2019.

(51) Int. Cl. A61K 38/17; A61K 48/00; A61P 17/14

(52) U.S. Cl. CPC A61K 38/1709; A61K 48/0008; A61P 17/14

(57) ABSTRACT

Disclosed herein are methods for modulating hair growth and increasing hair follicle stem cell activation in an individual in need thereof. This includes administering an agent that modulates a Gas6-Tyro3-Axl-Mertk (TAM) interaction or pathway.

### RELATED APPLICATIONS

This application is related to and claims the benefit of U.S. Provisional Application No. 62/932,501, filed Nov. 7, 2019. The entire teachings of the applications are incorporated herein by reference.

### BACKGROUND OF THE INVENTION

Chronic exposure to stressors can profoundly impact tissue homeostasis and regeneration. However, how stress leads to tissue changes remains largely elusive.

### SUMMARY OF THE INVENTION

A pathway mediated by Gas6 has been identified as shown herein as potent in promoting hair follicle stem cell (HFSCs) activation and hair growth under both normal and stress conditions. Moreover, AXL, an interaction partner for Gas6, has been identified as described below as involved in hair follicle stem cell (HFSCs) activation and hair growth; the AXL inhibitor R428 is shown herein to inhibit hair growth. Work described herein reveals an unprecedented regenerative capacity of mouse HFSCs when released from the systemic control of corticosterone, increasing Gas6-mediated HFSC activation.

Some aspects of the disclosure are related to methods of modulating hair growth, methods of modulating HFSC activation, methods of modulating HFSC quiescence and/or methods of modulating hair follicle regeneration or activation in an individual in need thereof.

The described methods include administering an agent that modulates a Gas6-Tyro3/Axl/Mertk (TAM) interaction or pathway. In some embodiments, the TAM interaction or pathway is an AXL, a Tyro3, or a Mertk interaction or pathway. In some embodiments the agent is a corticosterone modulator (i.e., inhibitor or enhancer).

In some embodiments, the agent modulates Gas6 activity or expression. In some embodiments, the agent increases Gas6 activity or expression. In some embodiments the agent is Gas6, and in some embodiments the Gas6 is administered or delivered via an AAV vector.

In some embodiments, the agent decreases Gas6 activity or expression. In some embodiments the agent modulates the interaction of Gas6 with AXL. In some embodiments the agent modulates the interaction of Gas6 with Tyro3. In some embodiments the agent modulates the interaction of Gas6 with Mertk.

In some embodiments, the agent increases hair growth or HFSC activation. In some embodiments, the agent decreases hair growth or HFSC activation. In some embodiments, the method increases hair growth or HFSC activation. In some embodiments, the method increases hair growth or HFSC activation by at least 5%, at least 10%, at least 15%, at least 20% or at least 25% relative to a suitable control. In some embodiments, the method decreases hair growth or HFSC activation. In some embodiments, the method decreases hair growth or HFSC activation by at least 5%, at least 10%, at least 15%, at least 20% or at least 25% relative to a suitable control.

In some embodiments, the method increases hair growth or HFSC activation under stress conditions. In some embodiments, the stress condition is characterized by elevated corticosterone levels or by hair loss or a hair loss condition. In some embodiments, the hair loss condition is telogen effluvium.

In some embodiments, the agent is administered using an AAV vector. In some embodiments, the AAV is AAV8. In some embodiments, the agent is administered through intradermal injection.

In some embodiments, the agent modulates AXL activity or expression. In some embodiments, the agent increases AXL activity or expression. In some embodiments, the agent decreases AXL activity or expression. In particular embodiments the agent interferes with or inhibits, partly or fully, the interaction between Gas6 and AXL.

The practice of the present invention will typically employ, unless otherwise indicated, conventional techniques of cell biology, cell culture, molecular biology, transgenic biology, microbiology, recombinant nucleic acid (e.g., DNA) technology, immunology, and RNA interference (RNAi) which are within the skill of the art. Non-limiting descriptions of certain of these techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., edition as of December 2008; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Antibodies—A Laboratory Manual, Cold Spring Harbor Laboratory Press, Cold Spring Harbor, 1988; Freshney, R. I., “Culture of Animal Cells, A Manual of Basic Technique”, 5th ed., John Wiley & Sons, Hoboken, N.J., 2005. Non-limiting information regarding therapeutic agents and human diseases is found in Goodman and Gilman's The Pharmacological Basis of Therapeutics, 11th Ed., McGraw Hill, 2005, Katzung, B. (ed.) Basic and Clinical Pharmacology, McGraw-Hill/Appleton & Lange; 10th ed. (2006) or 11th edition (July 2009). Non-limiting information regarding genes and genetic disorders is found in McKusick, V. A.: Mendelian Inheritance in Man. A Catalog of Human Genes and Genetic Disorders. Baltimore: Johns Hopkins University Press, 1998 (12th edition) or the more recent online database: Online Mendelian Inheritance in Man, OMIM™. McKusick-Nathans Institute of Genetic Medicine, Johns Hopkins University (Baltimore, Md.) and National Center for Biotechnology Information, National Library of Medicine (Bethesda, Md.), as of May 1, 2010, ncbi.nlm.nih.gov/omim/ and in Online Mendelian Inheritance in Animals (OMIA), a database of genes, inherited disorders and traits in animal species (other than human and mouse), at omia.angis.org.au/contact.shtml. All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed features and attendant advantages of the present inventions will become better understood by reference to the following detailed description of the invention in conjunction with the attached drawings.

### BRIEF DESCRIPTION OF THE DRAWINGS

FIGS. 1A-1J demonstrate removal of adrenal glands leads to loss of quiescence in HFSCs. FIG. 1A provides a schematic of the hair cycle in sham-operated (Sham) and adrenalectomized (ADX) mice. Telo, telogen; Ana, anagen. FIG. 1B shows Sham and ADX are shaved and monitored for hair coat recovery (indicative of anagen). Quantifications show % of back skin covered by regrown hairs after shaving at P43 (Sham: n=5, ADX: n=4; P=0.0498 at P66, P<0.0001 at P74 and P81). FIG. 1C shows H&E of P74 Sham and ADX skins. FIG. 1D shows immunocolocalization of EdU and PCAD in P56 Sham and ADX hair follicles. FIG. 1E provides a comparison of EdU localization in bulge and upper outer root sheath in late anagen Sham and ADX mice (Sham, P109; ADX, P67). FIG. 1F provides quantification of 3rd telogen length (Sham, n=4; ADX, n=6). FIG. 1G provides quantification showing the number of hair cycles of Sham (n=7) and ADX mice (n=5) from P60 to P444 (see also FIG. 6). FIG. 1H shows representative hair regeneration status of 18-month-old Sham and ADX mice. FIG. 1I shows H&E of Sham and ADX skins at 21 months old. FIG. 1J provides representative hair regeneration status of 22-month-old Sham and ADX mice (left). Quantification of HFSC number per hair follicle in Sham (n=7) and ADX (n=8) (middle). Quantification of % of HFSCs in epithelial fraction by FACS in Sham and ADX (n=2 for each) (right). Yellow lines: bulge, white lines: hair follicle. Scale bars, 50 μm. Data are mean±SEM, two-tailed t-test.

FIGS. 2A-2H demonstrate corticosterone secreted from the adrenal gland regulates HFSC quiescence. FIG. 2A shows hormones from the adrenal gland. FIG. 2B shows plasma levels of corticosterone in P45 Sham and ADX (n=3 for each). FIG. 2C provides experimental design for vehicle or corticosterone feeding. FIG. 2D provides plasma corticosterone levels in sham fed with vehicle, ADX with vehicle, and ADX with corticosterone 2 weeks after feeding (n=3 for each). FIG. 2E shows hair cycle progression in Sham+Veh (n=4), ADX+Veh (n=3), and ADX+CORT (n=4) (Sham+Veh versus ADX+Veh: P=0.0335 at P66, P<0.0001 at P73; ADX+Veh versus ADX+CORT: P=0.0387 at P66, P<0.0001 at P73). FIG. 2F provides representative H&E of P73 Sham+Vehicle, ADX+Vehicle, and ADX+Corticosterone. FIG. 2G shows hair cycle progression of C57BL/6 mice fed with vehicle or corticosterone (n=5 for each; P=0.0125 (P94), 0.0019 (P97), 0.0023 (P100), 0.0015 (P104), 0.0011 (P107), 0.0001 (P111), <0.0001 (P118)). FIG. 2H shows hair cycle progression in C57BL/6 mice subjected to chronic unpredictable stress (n=10) and non-stressed control (n=12). P=0.0494 (P94), 0.0268 (P97), 0.0152 (P100), 0.0057 (P104), 0.0017 (P107), 0.0012 (P111), 0.0012 (P118). Veh, vehicle; CORT, corticosterone. Data are mean±SEM, two-tailed t-test.

FIGS. 3A-3G demonstrate corticosterone acts on the dermal niche to regulate HFSC quiescence. FIG. 3A provides a schematic of K15-CrePGR activity and qRT-PCR of GR from FACS-purified HFSCs of control or K15-CrePGR; GR fl/fl (n=3 for each). FIG. 3B shows hair cycle progression of control and K15-CrePGR GR fl/fl mice and quantifications (n=3 for each; P=0.2767 at P148). FIG. 3C provides a schematic of Pdgfra-CreER activity and qRT-PCR of GR from FACS-purified dermal papilla (DP) and dermal fibroblasts (DF) of control and Pdgfra-CreER; GR fl/fl (n=3 for each). FIG. 3D shows hair cycle progression of control and Pdgfra-CreER; GR fl/fl mice and quantifications (n=3 for each; P=0.0211 at P94, P=0.0003 at P100). FIG. 3E provides a schematic of Sox2-CreER activity (blue) and immunohistochemical analyses (YFP and PCAD) of Sox2-CreER; R26-lsl-YFP skin. Representative images showing YFP in DP of guard hair follicles, but not zigzag hair follicles, of Sox2-CreER; R26-lsl-YFP. FIG. 3F shows H&E of control (top) and Sox2-CreER; GR fl/fl skin (bottom), arrowhead denotes an anagen guard hair follicle surrounded by telogen zigzag hair follicles in Sox2-CreER GR fl/fl skin. FIG. 3G provides representative images of dorsal skin showing accelerated hair regeneration only in the guard hairs in Sox2-CreER GR fl/fl mice (shaved at P45 and image taken at P67). Scale bars, 50 μm. Data are mean±SEM, two-tailed t-test.

FIGS. 4A-4G demonstrate secretome analyses identified Gas6 as a secreted factor suppressed by systemic corticosterone in the dermal papilla. FIGS. 4A-4B provides experimental workflow and principal component analysis (PCA) comparing the transcriptome of DP cells FACS-purified from Sham and ADX (in FIG. 4A) or control and Pdgfra-CreER GR fl/fl (in FIG. 4B). FIG. 4C shows secretome analyses identifying common secreted factors from the differentially expressed genes (DEGs, >1.5 fold, P<0.05) in ADX and Pdgfra-CreER GR fl/fl DP cells. FIGS. 4D-4E shows expression levels of common differentially expressed secreted factors as transcripts per million (TPM), in the DP cells of ADX (FIG. 4D) and Pdgfra-CreER GR fl/fl mice (FIG. 4E). Data are mean±SEM. Adjusted P values are calculated by the Benjamini-Hochberg procedure in DESeq2. FIG. 4F provides schematics of the Gas6/Axl receptor tyrosine kinase. R428 is a selective inhibitor of Axl tyrosine kinase activity (left). Colony formation assays of cultured HFSCs in the presence of Gas6 (middle) or R428 (right). FIG. 4G shows hair cycle progression of Sham and ADX mice treated with ethanol topically (P=0.0154 at P60, P=0.0115 at P66, P<0.0001 at P73), or ADX mice treated with R428 in ethanol (P=0.0266 at P60, P=0.0206 at P66, P=0.0238 at P73) (n=4 for each). EtOH, Ethanol. Data are mean±SEM, two-tailed t-test (FIG. 4f, 4g).

FIGS. 5A-5H demonstrate Gas6 overexpression counteracts the inhibitory effect of corticosterone under stress. FIG. 5A provides a schematic and experimental design of intradermal AAV injections. AAV-GFP is used as a control. FIGS. 5B-5C show injection of AAV-Gas6 into the centre of the telogen dorsal skin induces anagen in the injected regions. Dashed circles indicate AAV injection areas. Scale bar, 50 μm. FIG. 5D shows qRT-PCR of Gas6 from FACS-purified DP cells of control and stressed mice (n=3 for each). Data are mean±SEM, two-tailed t-test. FIGS. 5E-5G show AAV-mediated expression of GFP (control) or Gas6 in mice subjected to chronic unpredictable stress (FIG. 5F) or chronic corticosterone feeding (FIG. 5G). FIG. 5H provides a model summarizing main findings. Corticosterone secreted from the adrenal gland modulates HFSC activity through regulating Gas6 expression in the DP. When corticosterone levels are increased under stress, Gas6 expression is inhibited, HFSCs remain quiescent. By contrast, when corticosterone levels decrease, elevated Gas6 promotes HFSC activation.

FIGS. 6A-6B demonstrate hair cycle progression in control and ADX mice long-term. FIG. 6A shows representative hair regeneration status of Sham and ADX mice from P60 to P444. To observe new hair growth, the mice are shaved as soon as the newly regenerated hairs cover >90% of the back skin. FIG. 6B provides immunohistochemical analyses (CD34 and PCAD) of telogen hair follicle in Sham and ADX skins at 22-month-old showing normal hair follicle morphology and comparable stem cell numbers. Yellow lines: bulge. White lines: hair germ. Scale bars, 50 μm.

FIG. 7 demonstrates the levels of norepinephrine and epinephrine in Sham and ADX mice. Plasma levels of norepinephrine and epinephrine are measured by LC-MS/MS in P45 Sham and ADX mice (n=3 for each). Data are mean±SEM, two-tailed t-test.

FIGS. 8A-8F demonstrate corticosterone levels are elevated in stressed mice. FIG. 8A shows plasma corticosterone levels in C57BL/6 mice fed with vehicle or corticosterone (n=3 for each). Veh, vehicle; CORT, corticosterone. FIGS. 8B-8C provide representative H&E staining (FIG. 8B) and immunohistochemical analyses of active caspase 3 (aCAS3) and PCAD (FIG. 8C) in vehicle and corticosterone feeding mice. FIG. 8D shows plasma corticosterone levels in non-stressed control and mice subjected to chronic unpredictable stress (n=3 for each). FIGS. 8E-8F provide representative H&E staining (FIG. 8E) and immunohistochemical analyses of active caspase3 (aCAS3) and PCAD (FIG. 8F) in non-stressed control and stressed mice. Scale bars, 50 μm (FIG. 8C, FIG. 8F). Data are mean±SEM, two-tailed t-test.

FIGS. 9A-9B demonstrate removal of the adrenal glands suppress the effect of chronic stress on hair follicle regeneration. FIG. 9A provides an experimental timeline for chronic unpredictable stress on Sham and ADX mice. FIG. 9B shows stressed Sham (Sham+Stress) and stressed ADX (ADX+Stress) mice are monitored for hair coat recovery. Quantification shows % of back skin covered by newly regenerated hairs (Sham+Stress: n=5, ADX+Stress mice n=3; P=0.0123 at P66, P<0.0001 at P74 and P81). Data are mean±SEM, two-tailed t-test.

FIGS. 10A-10E demonstrate FACS strategies and hair cycle validation for transcriptome analyses in control, ADX, and dermal GR knockout mice. FIG. 10A shows FACS strategies for isolating DP cells for RNA-Seq^{41,46}. FIGS. 10B-10C provide immunohistochemical analyses (PCAD) for skin samples used in RNAseq experiments to validate hair cycle stages (telogen). In FIG. 10B, sham and ADX; in FIG. 10C, control and Pdgfra-CreER GR fl/fl skins. FIG. 10D provides a heatmap of differentially expressed genes (>1.5 fold, P<0.05) in Sham and ADX mice. FIG. 10E provides a heatmap of differentially expressed genes (>1.5 fold, P<0.05) in control and Pdgfra-CreER GR fl/fl mice.

FIGS. 11A-11B demonstrate TAM receptor levels in HFSCs. FIG. 11A provides a schematic of Gas6 and the TAM receptors (TYRO3, AXL, MERTK). FIG. 11B shows RNA-Seq read counts for the expression of TAM receptors (TYRO3, AXL, MERTK) in FACS-purified HFSCs. Data are mean±SEM.

FIGS. 12A-12D demonstrate intradermal injection of AAV-GFP and AAV-Gas6 enables transduction of dermal fibroblasts. FIG. 12A provides an experimental design of AAV-GFP injection. FIG. 12B provides representative immunohistochemical analyses (GFP and PCAD) of PBS-injected 2nd telogen skin (control, left) and AAV-GFP injected 2nd telogen skin (right). Scale bar, 50 μm. FIG. 12C provides an experimental design of AAV-Gas6 injection. FIG. 12D shows qRT-PCR of Gas6 gene from FACS-purified dermal fibroblasts of PBS-injected 2nd telogen skin (control) and AAV-Gas6 injected 2nd telogen skin (n=2 for each). Data are mean±SEM, two-tailed t-test.

FIGS. 13A-13B demonstrate dermal fibroblast-specific GR knockout suppresses the impact of chronic stress on hair follicle regeneration. FIGS. 13A-13B provide a schematic of experimental design (FIG. 13A). Control and Pdgfra-CreER GR fl/fl mice are subjected to chronic unpredictable stress from P55, and their hair cycle changes are monitored in (FIG. 13B). Quantification shows of % of hair re-growth at the back skin at P114 (control mice with stress n=2; Pdgfra-CreER GR fl/fl with Stress n=3). Data are mean±SEM, two-tailed t-test.

FIG. 14 demonstrates the sympathetic nerve is required for stress-induced hair graying.

FIG. 15 demonstrates how stem cells during hair cycles maintaining quiescence.

FIGS. 16A-16F demonstrate removal of adrenal gland leads to loss of HFSC quiescence. FIGS. 16A-16B show removal of adrenal gland significantly shorten the resting phase. FIGS. 16C-16D show ADX mice have changes in quiescence status in late anagen. FIG. 16E shows ADX mice continue to enter hair cycles. FIG. 16F shows older ADX mice display normal hair coat.

FIGS. 17A-17D demonstrates that stress hormones are crucial for regulating HFSC quiescence. FIGS. 17A-17B show the levels of corticosterone, norepinephrine, and epinephrine in Sham and ADX mice. FIGS. 17C-17D show feeding corticosterone (CORT) suppress the loss of HFSC quiescence of ADX mice.

FIG. 18 shows the hair growth cycle.

FIGS. 19A-19F demonstrate stress hormones regulate hair cycles through the dermal niche. FIGS. 19A-19B shows glucocorticoid receptor (GR) is conditionally deleted from the adult hair follicle stem cells. K15-CrePGR GR cKO mice do not show significant difference in telogen length. FIGS. 19C-19D show Pdgfra-CreER GRcKO display shorter telogen length and anagen entry. FIGS. 19E-19F show Sox2-CreER GRcKO mice display anagen entry in most guard hairs.

FIG. 20 shows the dermal niche factors.

FIGS. 21A-21C demonstrate that a candidate gene promotes HFSC activation. FIG. 21A shows Gas6-AXL pathway. FIG. 21B shows colony formation assay on control and Gas6-treated HFSCs. FIGS. 21C-21D show topical R428 treatment repress the phenotypes of ADX.

FIGS. 22A-22C demonstrate Gas6 rescue stress-induced HF regeneration failure. FIG. 22A shows HPA axis. FIG. 22B shows chronic stress mice show delayed HF regeneration. FIG. 22C shows adeno-associated virus (AAV)-mediated Gas6 expression induce HF regeneration to chronic stress mice.

FIG. 23 shows the stress hormone is an essential factor in maintaining the quiescence of hair follicle stem cell by regulating the dermal niche for the proper stem cell homeostasis.

### DETAILED DESCRIPTION OF THE INVENTION

The present invention reveals that the adrenal gland-derived stress hormone corticosterone (the cortisol equivalent in rodents) enforces hair follicle stem cell quiescence in mice. Without corticosterone, hair follicle stem cells lose quiescence and enter continuous rounds of regeneration cycles throughout life with no signs of exhaustion. Conversely, under chronic stress, elevated corticosterone levels prolong hair follicle stem cell quiescence and inhibit hair follicle regeneration. Mechanistically, corticosterone acts on the dermal niche to suppress the expression of Growth Arrest Specific 6 (Gas6), a secreted factor that stimulates hair follicle stem cell activation. Restoring Gas6 expression levels is sufficient to overcome the stress-induced regeneration block on hair follicle stem cells. The findings delineate a cellular and molecular mechanism by which stress leads to defects in tissue regeneration and methods by which such defects can be prevented to therapeutic effect. Moreover, corticosterone is identified as a potent systemic inhibitor of hair follicle stem cell activity via its impact on the niche, and removal of such inhibition drives hair follicle stem cells into continuous regeneration cycles without losing stem cell potential.

Disclosed herein are methods for modulating hair growth and increasing hair follicle stem cell activation in an individual in need thereof. This includes administering an agent that modulates a Gas6-Tyro3/Axl/Mertk (TAM) interaction or pathway.

Stem cell quiescence and activation dictate the timing, frequency, and amount of tissue production. Seminal studies have established that stem cell quiescence is governed by intrinsic regulators as well as extrinsic signals from the niche¹⁻⁶. However, tissue production and homeostasis differ substantially in different physiological states. For example, chronic, sustained stress is thought to cause profound defects in regeneration processes. How systemic factors regulate stem cell quiescence and activation to couple tissue regeneration with diverse bodily changes remains to be determined.

Methods of Modulating Hair Growth and Hair Follicle Stem Cell Activation

Aspects of the disclosure are related to methods of modulating hair growth and HFSC activation in an individual in need thereof. This includes administering an agent that modulates a Gas6-Tyro3/Axl/Mertk (TAM) interaction or pathway (e.g., turns the pathway on or off). In some embodiments, the TAM interaction or pathway is an AXL, a Tyro3, or a Mertk interaction or pathway.

As used herein, “modulating” or “modulates” means causing or facilitating a qualitative or quantitative change, alteration, or modification. Without limitation, such change may be an increase or decrease in a qualitative or quantitative aspect. For example, modulating transcription of a gene includes increasing or decreasing the rate or frequency of gene transcription.

As used herein, “agent” broadly refers to any substance, compound (e.g., molecule), supramolecular complex, material, or combination or mixture thereof. In some aspects, an agent can be represented by a chemical formula, chemical structure, or sequence. Examples of agents, include, e.g., small molecules, polypeptides, nucleic acids (e.g., RNAi agents, antisense oligonucleotide, aptamers), lipids, polysaccharides, peptide mimetics, analogs, etc. In some embodiments an agent may be a gene editing agent (e.g., CRISPR/Cas9, TALEN, ZFN, etc.). In general, agents may be obtained using any suitable method known in the art. The ordinary skilled artisan will select an appropriate method based, e.g., on the nature of the agent. An agent may be at least partly purified. In some embodiments an agent may be provided as part of a composition, which may contain, e.g., a counter-ion, aqueous or non-aqueous diluent or carrier, buffer, preservative, or other ingredient, in addition to the agent, in various embodiments. In some embodiments an agent may be provided as a salt, ester, hydrate, or solvate. In some embodiments an agent is cell-permeable, e.g., within the range of typical agents that are taken up by cells and acts intracellularly, e.g., within mammalian cells. Certain compounds may exist in particular geometric or stereoisomeric forms. Such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof are encompassed by this disclosure in various embodiments unless otherwise indicated. Certain compounds may exist in a variety of protonation states, may have a variety of configurations, may exist as solvates (e.g., with water (i.e. hydrates) or common solvents) and/or may have different crystalline forms (e.g., polymorphs) or different tautomeric forms. Embodiments exhibiting such alternative protonation states, configurations, solvates, and forms are encompassed by the present disclosure where applicable.

In some embodiments, the agent is a nucleic acid. The term “nucleic acid” refers to polynucleotides such as deoxyribonucleic acid (DNA) and ribonucleic acid (RNA). The terms “nucleic acid” and “polynucleotide” are used interchangeably herein and should be understood to include double-stranded polynucleotides, single-stranded (such as sense or antisense) polynucleotides, and partially double-stranded polynucleotides. A nucleic acid often comprises standard nucleotides typically found in naturally occurring DNA or RNA (which can include modifications such as methylated nucleobases), joined by phosphodiester bonds. In some embodiments a nucleic acid may comprise one or more non-standard nucleotides, which may be naturally occurring or non-naturally occurring (i.e., artificial; not found in nature) in various embodiments and/or may contain a modified sugar or modified backbone linkage. Nucleic acid modifications (e.g., base, sugar, and/or backbone modifications), non-standard nucleotides or nucleosides, etc., such as those known in the art as being useful in the context of RNA interference (RNAi), aptamer, CRISPR technology, polypeptide production, reprogramming, or antisense-based molecules for research or therapeutic purposes may be incorporated in various embodiments. Such modifications may, for example, increase stability (e.g., by reducing sensitivity to cleavage by nucleases), decrease clearance in vivo, increase cell uptake, or confer other properties that improve the translation, potency, efficacy, specificity, or otherwise render the nucleic acid more suitable for an intended use. Various non-limiting examples of nucleic acid modifications are described in, e.g., Deleavey G F, et al., Chemical modification of siRNA. Curr. Protoc. Nucleic Acid Chem. 2009; 39:16.3.1-16.3.22; Crooke, S T (ed.) Antisense drug technology: principles, strategies, and applications, Boca Raton: CRC Press, 2008; Kurreck, J. (ed.) Therapeutic oligonucleotides, RSC biomolecular sciences. Cambridge: Royal Society of Chemistry, 2008; U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 6,455,308 and/or in PCT application publications WO 00/56746 and WO 01/14398. Different modifications may be used in the two strands of a double-stranded nucleic acid. A nucleic acid may be modified uniformly or on only a portion thereof and/or may contain multiple different modifications. Where the length of a nucleic acid or nucleic acid region is given in terms of a number of nucleotides (nt) it should be understood that the number refers to the number of nucleotides in a single-stranded nucleic acid or in each strand of a double-stranded nucleic acid unless otherwise indicated. An “oligonucleotide” is a relatively short nucleic acid, typically between about 5 and about 100 nt long. In some embodiments, the nucleic acid codes for MFSD12 or functional variants thereof.

As used herein, the term “RNAi agent” encompasses nucleic acids that can be used to achieve RNAi in eukaryotic cells. Short interfering RNA (siRNA), short hairpin RNA (shRNA), and microRNA (miRNA) are examples of RNAi agents. siRNAs typically comprise two separate nucleic acid strands that are hybridized to each other to form a structure that contains a double stranded (duplex) portion at least 15 nt in length, e.g., about 15-about 30 nt long, e.g., between 17-27 nt long, e.g., between 18-25 nt long, e.g., between 19-23 nt long, e.g., 15, 16, 17, 18, 19, 20, 21, 22, 23, 24, 25, 26, 27, 28, 29, or 30 nucleotides. In some embodiments the strands of an siRNA are perfectly complementary to each other within the duplex portion. In some embodiments the duplex portion may contain one or more unmatched nucleotides, e.g., one or more mismatched (non-complementary) nucleotide pairs or bulged nucleotides. In some embodiments either or both strands of an siRNA may contain up to about 1, 2, 3, or 4 unmatched nucleotides within the duplex portion. In some embodiments a strand may have a length of between 15-35 nt, e.g., between 17-29 nt, e.g., 19-25 nt, e.g., 21-23 nt. Strands may be equal in length or may have different lengths in various embodiments. In some embodiments strands may differ by 1-10 nt in length. A strand may have a 5′ phosphate group and/or a 3′ hydroxyl (—OH) group. Either or both strands of an siRNA may comprise a 3′ overhang of, e.g., about 1-10 nt (e.g., 1-5 nt, e.g., 2 nt). Overhangs may be the same length or different in lengths in various embodiments. In some embodiments an overhang may comprise or consist of deoxyribonucleotides, ribonucleotides, or modified nucleotides or modified ribonucleotides such as 2′-O-methylated nucleotides, or 2′-O-methyl-uridine. An overhang may be perfectly complementary, partly complementary, or not complementary to a target RNA in a hybrid formed by the guide strand and the target RNA in various embodiments.

shRNAs are nucleic acid molecules that comprise a stem-loop structure and a length typically between about 40-150 nt, e.g., about 50-100 nt, e.g., about 60-80 nt. A “stem-loop structure” (also referred to as a “hairpin” structure) refers to a nucleic acid having a secondary structure that includes a region of nucleotides which are known or predicted to form a double strand (stem portion; duplex) that is linked on one side by a region of (usually) predominantly single-stranded nucleotides (loop portion). Such structures are well known in the art and the term is used consistently with its meaning in the art. A guide strand sequence may be positioned in either arm of the stem, i.e., 5′ with respect to the loop or 3′ with respect to the loop in various embodiments. As is known in the art, the stem structure does not require exact base-pairing (perfect complementarity). Thus, the stem may include one or more unmatched residues or the base-pairing may be exact, i.e., it may not include any mismatches or bulges. In some embodiments the stem is between 15-30 nt, e.g., between 17-29 nt, e.g., between 19-25 nt. In some embodiments the stem is between 15-19 nt. In some embodiments the stem is between 19-30 nt. The primary sequence and number of nucleotides within the loop may vary. Examples of loop sequences include, e.g., UGGU; ACUCGAGA; UUCAAGAGA. In some embodiments a loop sequence found in a naturally occurring miRNA precursor molecule (e.g., a pre-miRNA) may be used. In some embodiments a loop sequence may be absent (in which case the termini of the duplex portion may be directly linked). In some embodiments a loop sequence may be at least partly self-complementary. In some embodiments the loop is between 1 and 20 nt in length, e.g., 1-15 nt, e.g., 4-9 nt. The shRNA structure may comprise a 5′ or 3′ overhang. As known in the art, an shRNA may undergo intracellular processing, e.g., by the ribonuclease (RNase) III family enzyme known as Dicer, to remove the loop and generate an siRNA.

Mature endogenous miRNAs are short (typically 18-24 nt, e.g., about 22 nt), single-stranded RNAs that are generated by intracellular processing from larger, endogenously encoded precursor RNA molecules termed miRNA precursors (see, e.g., Bartel, D., Cell. 116(2):281-97 (2004); Bartel D P. Cell. 136(2):215-33 (2009); Winter, J., et al., Nature Cell Biology 11: 228-234 (2009). Artificial miRNA may be designed to take advantage of the endogenous RNAi pathway in order to silence a target RNA of interest. The sequence of such artificial miRNA may be selected so that one or more bulges is present when the artificial miRNA is hybridized to its target sequence, mimicking the structure of naturally occurring miRNA:mRNA hybrids. Those of ordinary skill in the art are aware of how to design artificial miRNA.

An RNAi agent that contains a strand sufficiently complementary to an RNA of interest so as to result in reduced expression of the RNA of interest (e.g., as a result of degradation or repression of translation of the RNA) in a cell or in an in vitro system capable of mediating RNAi and/or that comprises a sequence that is at least 80%, 90%, 95%, or more (e.g., 100%) complementary to a sequence comprising at least 10, 12, 15, 17, or 19 consecutive nucleotides of an RNA of interest may be referred to as being “targeted to” the RNA of interest. An RNAi agent targeted to an RNA transcript may also be considered to be targeted to a gene from which the transcript is transcribed.

In some embodiments an RNAi agent is a vector (e.g., an expression vector) suitable for causing intracellular expression of one or more transcripts that give rise to a siRNA, shRNA, or miRNA in the cell. Such a vector may be referred to as an “RNAi vector”. An RNAi vector may comprise a template that, when transcribed, yields transcripts that may form a siRNA (e.g., as two separate strands that hybridize to each other), shRNA, or miRNA precursor (e.g., pri-miRNA or pre-mRNA).

An RNAi agent may be produced in any of a variety of ways in various embodiments. For example, nucleic acid strands may be chemically synthesized (e.g., using standard nucleic acid synthesis techniques) or may be produced in cells or using an in vitro transcription system. Strands may be allowed to hybridize (anneal) in an appropriate liquid composition (sometimes termed an “annealing buffer”). An RNAi vector may be produced using standard recombinant nucleic acid techniques.

In some embodiments, the agent is a small molecule. The term “small molecule” refers to an organic molecule that is less than about 2 kilodaltons (kDa) in mass. In some embodiments, the small molecule is less than about 1.5 kDa, or less than about 1 kDa. In some embodiments, the small molecule is less than about 800 daltons (Da), 600 Da, 500 Da, 400 Da, 300 Da, 200 Da, or 100 Da. Often, a small molecule has a mass of at least 50 Da. In some embodiments, a small molecule is non-polymeric. In some embodiments, a small molecule is not an amino acid. In some embodiments, a small molecule is not a nucleotide. In some embodiments, a small molecule is not a saccharide. In some embodiments, a small molecule contains multiple carbon-carbon bonds and can comprise one or more heteroatoms and/or one or more functional groups important for structural interaction with proteins (e.g., hydrogen bonding), e.g., an amine, carbonyl, hydroxyl, or carboxyl group, and in some embodiments at least two functional groups. Small molecules often comprise one or more cyclic carbon or heterocyclic structures and/or aromatic or polyaromatic structures, optionally substituted with one or more of the above functional groups.

In some embodiments, the agent is a protein or polypeptide. The term “polypeptide” refers to a polymer of amino acids linked by peptide bonds. A protein is a molecule comprising one or more polypeptides. A peptide is a relatively short polypeptide, typically between about 2 and 100 amino acids (aa) in length, e.g., between 4 and 60 aa; between 8 and 40 aa; between 10 and 30 aa. The terms “protein”, “polypeptide”, and “peptide” may be used interchangeably. In general, a polypeptide may contain only standard amino acids or may comprise one or more non-standard amino acids (which may be naturally occurring or non-naturally occurring amino acids) and/or amino acid analogs in various embodiments. A “standard amino acid” is any of the 20 L-amino acids that are commonly utilized in the synthesis of proteins by mammals and are encoded by the genetic code. A “non-standard amino acid” is an amino acid that is not commonly utilized in the synthesis of proteins by mammals. Non-standard amino acids include naturally occurring amino acids (other than the 20 standard amino acids) and non-naturally occurring amino acids. An amino acid, e.g., one or more of the amino acids in a polypeptide, may be modified, for example, by addition, e.g., covalent linkage, of a moiety such as an alkyl group, an alkanoyl group, a carbohydrate group, a phosphate group, a lipid, a polysaccharide, a halogen, a linker for conjugation, a protecting group, a small molecule (such as a fluorophore), etc.

In some embodiments, the agent is a peptide mimetic. The terms “mimetic,” “peptide mimetic” and “peptidomimetic” are used interchangeably herein, and generally refer to a peptide, partial peptide or non-peptide molecule that mimics the tertiary binding structure or activity of a selected native peptide or protein functional domain (e.g., binding motif or active site). These peptide mimetics include recombinantly or chemically modified peptides, as well as non-peptide agents such as small molecule drug mimetics.

In some embodiments, the agent is encoded by a synthetic RNA (e.g., modified mRNAs). The synthetic RNA can encode any suitable agent described herein. Synthetic RNAs, including modified RNAs are taught in WO 2017075406, which is herein incorporated by reference. In some embodiments, the agent is a synthetic RNA.

In some embodiments, the agent modulates Gas6 activity or expression. In some embodiments, the agent modulates the interaction of Gas6 with AXL. In some embodiments, the agent modulates the interaction of Gas6 with Tyro3. In some embodiments, the agent modulates the interaction of Gas6 with Mertk. In some embodiments, the agent (e.g., a chemical agent) modulates the Gas6-AXL pathway.

In some embodiments, the agent increases Gas6 activity or expression. In some embodiments, the agent increases Gas6 activity by at least 5%, at least 10%, at least 15%, at least 20%, or at least 25%, e.g., relative to a suitable control. In some aspects, the agent increases Gas6 activity by at least 5%. In some aspects, the agent increases Gas6 activity by at least 10%. In some aspects, the agent increases Gas6 activity by at least 15%. In some aspects, the agent increases Gas6 activity by at least 20%. In certain aspects, the agent increases Gas6 activity by at least 25%. In some embodiments, the agent decreases Gas6 activity or expression. In some embodiments, the agent decreases Gas6 activity by at least 5%, at least 10%, at least 15%, at least 20%, or at least 25%, e.g., relative to a suitable control. In some aspects, the agent decreases Gas6 activity by at least 5%. In some aspects, the agent decreases Gas6 activity by at least 10%. In some aspects, the agent decreases Gas6 activity by at least 15%. In some aspects, the agent decreases Gas6 activity by at least 20%. In certain aspects, the agent decreases Gas6 activity by at least 25%.

In some embodiments, the agent increases hair growth or HFSC activation. In some embodiments, the method increases hair growth or HFSC activation. In certain aspects, the method increases hair growth. In certain aspects, the method increases HFSC activation. In some embodiments, the method increases hair growth or HFSC activation relative to a suitable control. In some embodiments, the agent increases hair growth or HFSC activation by at least 5%, at least 10%, at least 15%, at least 20%, or at least 25%. In some aspects, the agent increases hair growth or HFSC activation by at least 5%. In some aspects, the agent increases hair growth or HFSC activation by at least 10%. In some aspects, the agent increases hair growth or HFSC activation by at least 15%. In some aspects, the agent increases hair growth or HFSC activation by at least 20%. In certain aspects, the agent increases hair growth by at least 25%. In certain embodiments, the agent increases HFSC activation by at least 25%.

In some embodiments, the method decreases hair growth or HFSC activation. In certain aspects, the method decreases hair growth. In certain aspects, the method decreases HFSC activation. In some embodiments, the method decreases hair growth or HFSC activation relative to a suitable control. In some embodiments, the agent decreases hair growth or HFSC activation by at least 5%, at least 10%, at least 15%, at least 20%, or at least 25%. In some aspects, the agent decreases hair growth or HFSC activation by at least 5%. In some aspects, the agent decreases hair growth or HFSC activation by at least 10%. In some aspects, the agent decreases hair growth or HFSC activation by at least 15%. In some aspects, the agent decreases hair growth or HFSC activation by at least 20%. In certain aspects, the agent decreases hair growth by at least 25%. In certain embodiments, the agent decreases HFSC activation by at least 25%.

In some embodiments, the method increases hair growth or HFSC activation under stress conditions. In some embodiments, the stress condition is characterized by elevated corticosterone or hair loss such as a hair loss condition. In some embodiments, the hair loss condition is telogen effluvium. As used herein, “telogen effluvium” refers to a form of temporary hair loss that usually happens after stress, a shock, or a traumatic event and occurs on the top of the scalp. Telogen effluvium is different from the permanent hair loss disorder called alopecia areata.

In some embodiments, the agent is administered using an AAV vector. In some embodiments, the AAV is selected from the group consisting of AAV1, AAV2, AAV3, AAV4, AAV5, AAV6, AAV7, AAV8, AAV9, AAV10, and AAV11. In certain embodiments, the AAV is AAV8.

The agents disclosed herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Exemplary modes of administration include, but are not limited to, injection, infusion, instillation, inhalation, or ingestion. “Injection” includes, without limitation, intravenous, intramuscular, intraarterial, intrathecal, intraventricular, intracranial, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In certain embodiments, the agent is administered through intradermal injection.

For topical application, the compositions and agents are formulated into solutions, suspensions, lotions, sprays, shampoos, hair conditions, serums, patches, wipes, gels, hydrogels, powders, patches, impregnated pads, emulsions, vesicular dispersions, sprays, aerosols, foams, ointments, tinctures, salves, gels, cleansing soaps, cleansing cakes, or creams as generally known in the art. The formulation can be, e.g., in a multi-use or single-use applicator. Topical administration can include the application of the pharmaceutical or cosmetic compositions to the scalp and/or hair.

The agent is not limited and may be any agent, or combination of agents, described herein or known in the art and suitable for the intended purpose or effect. In some embodiments, the agent comprises a polypeptide, amino acid, oligonucleotide, lipid, carbohydrate, hybrid molecule, peptide, nucleic acid, or small molecule.

An “effective amount” or “effective dose” of an agent (or composition containing such agent) refers to the amount sufficient to achieve a desired biological and/or pharmacological effect, e.g., when delivered to a cell or organism according to a selected administration form, route, and/or schedule. As will be appreciated by those of ordinary skill in this art, the absolute amount of a particular agent or composition that is effective may vary depending on such factors as the desired biological or pharmacological endpoint, the agent to be delivered, the target tissue, etc. Those of ordinary skill in the art will further understand that an “effective amount” may be contacted with cells or administered in a single dose, or through use of multiple doses, in various embodiments. A biological effect may be, e.g., reducing expression or activity of one or more gene products, reducing activity of a metabolic pathway or reaction, or reducing cell proliferation or survival of cells.

A single additional agent or multiple additional agents or treatment modalities may be co-administered (at the same or differing time points and/or via the same or differing routes of administration and/or on the same or a differing dosing schedule).

The dosage, administration schedule and method of administering the agent are not limited. In certain embodiments a reduced dose may be used when two or more agents are administered in combination either concomitantly or sequentially. The absolute amount will depend upon a variety of factors including other treatment(s), the number of doses and the individual patient parameters including age, physical condition, size and weight. These are factors well known to those of ordinary skill in the art and can be addressed with no more than routine experimentation. In some embodiments, a maximum tolerated dose may be used, that is, the highest safe and tolerable dose according to sound medical judgment.

As used herein, a “subject” means a human or animal (e.g., a mammal). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, minks, rabbits and hamsters. Domestic and game animals include cows, horses, pigs, deer, bison, buffalo, sheep, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e.g., a primate, e.g., a human. In some embodiments, where the subject is an animal (e.g., a sheep, mink, rabbit, etc.) fur growth may be modulated (i.e., increased or decreased) by modulating the Gas6/AXL pathway.

As used herein, pharmaceutical compositions comprise one or more agents or compositions that have therapeutic utility, and a pharmaceutically acceptable carrier, e.g., a carrier that facilitates delivery of agents or compositions. Agents and pharmaceutical compositions disclosed herein may be administered by any suitable means such as topically, orally, intranasally, intradermally, subcutaneously, intramuscularly, intravenously, intra-arterially, parenterally, intraperitoneally, intrathecally, intratracheally, ocularly, sublingually, vaginally, rectally, dermally, or as an aerosol.

In addition to the active agent(s), the pharmaceutical compositions typically comprise a pharmaceutically-acceptable carrier. The term “pharmaceutically-acceptable carrier”, as used herein, means one or more compatible solid or liquid vehicles, fillers, diluents, or encapsulating substances which are suitable for administration to a human or non-human animal. In preferred embodiments, a pharmaceutically-acceptable carrier is a non-toxic material that does not interfere with the effectiveness of the biological activity of the active ingredients. The term “compatible”, as used herein, means that the components of the pharmaceutical compositions are capable of being comingled with an agent, and with each other, in a manner such that there is no interaction which would substantially reduce the pharmaceutical efficacy of the pharmaceutical composition under ordinary use situations. Pharmaceutically-acceptable carriers should be of sufficiently high purity and sufficiently low toxicity to render them suitable for administration to the human or non-human animal being treated.

In some embodiments, the agent modulates AXL activity or expression. In some embodiments, the agent increases AXL activity or expression. In some embodiments, the agent decreases AXL activity or expression. Use of the AXL-specific inhibitor, R428, an anti-cancer drug candidate under clinical investigation, has been shown herein to inhibit hair growth. As used herein, “R428” inhibits the receptor tyrosine kinase AXL and induces apoptosis of many types of cancer cells. R428 is also known as BGB324 or bemcentinib. Several drugs classified as AXL inhibitors have entered clinical trials and are known in the art.

As used herein, “AXL” is a receptor tyrosine kinase (RTK) that was originally cloned from cancer cells. AXL belongs to a TAM (Tyro3, Axl and Mertk) family of RTKs. RTKs constitute a superfamily of transmembrane proteins that relays signals from extracellular growth factors into the cell. The TAM family receptors have in common a unique extracellular domain composed of two N-terminal immunoglobulin-like domains and two fibronectin type III repeats similar to the structure of neural cell adhesion molecules.

The TAM family of RTKs functions as homeostatic regulators in adult tissues and organ systems that are subject to continuous challenge and renewal throughout life. Their regulatory roles are prominent in the mature immune, reproductive, hematopoietic, vascular, and nervous systems. The TAMs and their ligands, Gas6 and Protein S, are essential for the efficient phagocytosis of apoptotic cells and membranes in these tissues. Deficiencies in TAM signaling may contribute to chronic inflammatory and autoimmune disease in humans, and aberrantly elevated TAM signaling is strongly associated with cancer progression, metastasis, and resistance to targeted therapies.

As used herein, “Gas6” means a gamma-carboxyglutamic acid-containing secreted protein which is the product of the growth arrest-specific gene 6. Gas6, cloned from serum-starved fibroblasts, is a member of the vitamin K-dependent family of Gla proteins homologous to the blood coagulation protein S.

Cortisol, commonly known as the “stress hormone”, is an adrenal gland-derived glucocorticoid which is up-regulated during stress in humans. In rodents, amphibians, and birds, the orthologous stress hormone secreted by adrenal glands is corticosterone, which possesses molecular features and functions equivalent to cortisol. Corticosterone plays diverse roles in organismal physiology. Under homeostatic conditions, baseline levels of corticosterone control blood sugar levels, regulate metabolism, and prevent inflammation⁷. Fear and acute stress stimuli trigger transient elevations of corticosterone as part of the fight-or-flight response, mobilizing energy and blocking processes not essential for immediate survival such as growth, the immune response, and reproduction⁸. Chronic stress prevents corticosterone levels from dissipating to baseline levels.

Physiological levels of corticosterone are known to influence cytokine-induced mobilization of haematopoietic stem cells from the bone marrow to the periphery⁹. Moreover, elevated corticosterone regulates neurogenesis in the hippocampus, affecting learning and memory in mice^{10,11}. The role of corticosterone in tissue regeneration, however, particularly its impact on stem cell quiescence and activation, remains largely unexplored. Thus, delineating the role and mechanisms by which corticosterone regulates tissue biology may provide critical insights toward understanding and potentially combating the detrimental impact of chronic stress.

The mouse hair follicle is an accessible and highly regenerative epithelial tissue well suited to study systemic regulation of stem cell quiescence. The hair follicle cycles between a resting phase (telogen) and a regeneration phase (anagen), where new hair follicles and hairs are generated¹². HFSCs are maintained in a quiescent state, except during early anagen, when HFSCs become transiently proliferative to initiate tissue regeneration¹³⁻¹⁶. Although anagen entry occurs regularly and synchronously in young mice (for the first two cycles), telogen length increases progressively over time, and anagen entry becomes rare and sporadic^{17,18}. Transcription factors, metabolic regulators, and secreted niche factors are known to influence HFSC quiescence and telogen length^{13,19-26}, and the hormone Prolactin promotes telogen during pregnancy^{27,28}. Beyond pregnancy, it is unclear whether a systemic regulator which normally controls the quiescent state of HFSCs exists. Since stress has been anecdotally associated with hair loss, and adrenalectomy has been found to accelerate hair growth in rats and minks^{29,30}, it was reasoned that adrenal gland-derived hormones might represent interesting candidates as systemic regulators of HFSCs.

By exploring the function of adrenal gland-derived corticosterone in regulating HFSCs in mice, corticosterone was identified as a potent systemic factor that enforces HFSC quiescence and telogen via suppressing a novel dermal niche secreted factor Gas6. The findings not only discover new regulators of HFSC quiescence and activation at both local and systemic levels, but also identify the cellular and molecular mechanisms by which chronic stress influences hair follicle regeneration. Moreover, it was demonstrated that the regeneration capacity of HFSCs remains even upon constant cycling. Therefore, it is possible to exploit HFSCs' remarkable potential to promote hair follicle regeneration through modulating the cortico sterone-Gas6 axis.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, cell, or non-human transgenic animal, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

The invention will be further described by the following non-limiting examples.

### EXAMPLES

### Materials and Methods

### Animals

C57BL/6J, GR flox⁷¹, K15-CrePGR¹⁵, Pdgfra-CreER⁷², Sox2-CreER⁷³, and R26-lsl-YFP⁷⁴ mice were from the Jackson Laboratory. The mice were maintained in an Association for Assessment and Accreditation of Laboratory Animal Care-approved animal facility at Harvard University. All procedures were approved by the Institutional Animal Care and Use Committee.

### Adrenalectomy

C57BL/6J mice were anesthetized. Small incisions were made on the back skin right on top of each adrenal gland. Both adrenal glands were removed with a pair of curved forceps. Sham-operated mice (Sham) underwent the same procedures as the ADX mice, except their adrenal glands were not removed.

### Chemical and Viral Treatment

RU486 (TCI America, Cat #M1732; 4% in ethanol) was used to induce K15-CrePGR by topical application for 10-14 days. To induce Pdgfra-CreER and Sox2-CreER, tamoxifen (Millipore Sigma, T5648; 20 mg/kg) was injected into mice intraperitoneally once per day for 4 to 6 days. To inhibit AXL activity, R428 (APExBIO, A8329; 2 mM in ethanol) was applied to ADX mice topically once a day. EdU (Lumiprobe Corporation, Cat #10540; 25 mg/kg) were administered by intraperitoneal injections. AAVs were produced as described previously⁴⁶ and injected directly into dermis through intradermal injections. 2-month-old C57BL/6J mice were injected with AAV-GFP or AAV-Gas6 (1×10¹¹ genome copy number per animal).

### Hair Cycle Analysis

The hair cycle progression was documented by standardized photographs at the start of each experiment and weekly thereafter. Anagen was determined by darkening of the skin followed by hair growth as previously described⁷⁵. The back skin of mice was shaved with an electric clipper to reveal skin colour changes and hair coat recovery. Once the hair coat recovery reached ˜90% of the back skin, the mice were shaved again to monitor the entry into next anagen. Telogen length was quantified as described previously¹⁹.

### Chronic Corticosterone Feeding

35 μg/ml Corticosterone (Millipore Sigma, C2505) was dissolved in vehicle (0.45% hydroxypropyl-β-cyclodextrine in drinking water) in drinking water during the entire corticosterone feeding period. Corticosterone water was changed every 3 days to prevent degradation. Control animals received the vehicle water.

### Chronic Unpredictable Stress

Chronic unpredictable stress (CUS) was adapted from protocols described previously^{35,36}. C57BL/6 mice, Sham, ADX, control mice, and Pdgfra-CreER GR fl/fl mice were exposed to diverse stressors according to the CUS timetable for 9 weeks. Two of the stressors were applied each day in a randomized fashion to prevent habituation. The stressors included cage tilt, isolation, crowding, damp bedding, rapid light-dark changes, overnight illumination, restraining, empty cage, and 3× cage changes.

### ELISA

Blood corticosterone levels were measured by ELISA (ARBOR assays, K014-H1) according to the manufacturer's instruction. Serum was collected using the heparinized tubes (Microvette® CB 300 LH or Microvette® 300 LH, Sarstedt) between 10 a.m. and 12 p.m.

### Liquid Chromatography-Tandem Mass Spectrometry

Blood epinephrine and norepinephrine were measured by liquid chromatography-tandem mass spectrometry (LC/MS/MS). The stable isotope labelled internal standards (d6-epinephrine, Cambridge Isotope Laboratories Inc.) was used for absolute quantification. The standards for the HPLC system were prepared using the catecholamine mixture (epinephrine and norepinephrine) (Millipore Sigma, C-109). All samples were carried out on an Agilent 6460 Triple Quadrupolo with an Agilent 1290 Infinity LC system.

### Histology and Immunohistochemistry

The skin samples were fixed in 4% paraformaldehyde (PFA, Electron Microscopy Sciences, Cat #15713) for 15 min at room temperature, washed in phosphate buffered saline (PBS), immersed in 30% sucrose solution overnight at 4° C., and embedded in optimal cutting temperature compound (Sakura Finetek, Cat #4583). 35 μm thick sections were fixed in 4% PFA. The fixed slides were then blocked in blocking buffer (5% Donkey serum, 1% bovine serum albumin, 2% cold-water fish gelatin in 0.3% Triton X-100 in PBS) for 1 h at room temperature, incubated with primary antibodies overnight at 4° C., and incubated with secondary antibodies for 2-4 h at room temperature. The following antibodies and dilutions were used: CD34 (eBioscience, 14-0341-85, 1:100), P-Cadherin (R&D Systems, AF761, 1:400), GFP (Abcam, ab290, 1:5000) and Cleaved Caspase-3 (Cell Signaling Technology, 1:300). DAPI was used as a counterstain for the nucleus. Cell proliferation assay was performed using a Click-It EdU Proliferation kit (Thermo Fisher Scientific, C10337) according to the manufacturer's instructions. Hematoxylin and eosin (H&E) staining was performed according standard protocols.

### Fluorescence-Activated Cell Sorting (FACS)

Dermal papilla cells and dermal fibroblasts were isolated as described^{41,46}. Briefly, mouse dorsal skin was dissected and treated with collagenase in Hank's Balanced Salt Solution for 20-30 min at 37° C. on an orbital shaker. The dermal fraction was collected by scraping followed by centrifugation at 300 g for 10 min. Dermal single-cell suspensions were obtained after 0.25% trypsin treatment for 10-20 min at 37° C. followed by filtering and centrifugation. Samples were stained for 30 min on ice. The following antibodies were used: Pdgfra-biotin (eBioscience, 13-1401-82; 1:250), CD45-eflour450 (eBioscience, 48-0451-82; 1:250), CD31-PE-Cy7 (eBioscience, 25-0311-81; 1:200), Sca-1-PerCP-Cy5.5 (eBiosciences, 45-5981-82, 1:1000), CD24-FITC (eBioscience, 11-0242-82; 1:250), and Streptavidin-APC (eBioscience, 17-4317-82; 1:500). DAPI was used to exclude dead cells. DP cells were isolated as CD45⁻, CD31⁻, Pdgfra⁺, CD24⁻, Sca-1⁻ cells.

For the isolation of HFSCs, mouse dorsal skin was dissected, and the fat layer was removed using a surgical scalpel. The skin was incubated in trypsin-EDTA at 37° C. for 35-45 min on an orbital shaker. Single-cell suspension was obtained by scraping the epidermal side and filtering. Cells were centrifuged for 8 min at 350 g at 4° C., resuspended in 5% fetal bovine serum, and stained for 30-40 min. The following antibodies were used: CD49f (Integrin alpha 6)-PE (eBioscience, 12-0495-82; 1:500); CD34-eFlour660 (eBioscience, 50-0341-82; 1:100); Sca-1-PerCP-Cy5.5 (eBioscience, 45-5981-82; 1:1000); and CD45-eFlour450 (eBioscience, 48-0451-82; 1:250). The HFSCs were isolated as CD45⁻, Integrin alpha 6⁺, CD34⁺, Sca-1⁻ cells.

### RNA Isolation

FACS-isolated cell populations were sorted directly into TRIzol LS Reagent (Thermo Fisher Scientific, Cat #10296028). RNA was isolated using an RNeasy Micro Kit (Qiagen, Cat #74004), using QIAcube according to the manufacturer's instructions. RNA concentration and RNA integrity were determined by Bioanalyzer (Agilent) using the RNA 6000 Pico kit (Agilent, Cat #5067-1513). High-quality RNA samples with RNA integrity number≥8 were used as input for qRT-PCR and RNA-sequencing.

### Complementary DNA Synthesis and Quantitative Real-Time PCR

Complementary DNA was synthesized using the Superscript IV VILO Master Mix with ezDNase Enzyme (Thermo Fisher Scientific, Cat #11766050). Quantitative real-time PCR was performed using power SYBR Green dye (Thermo Fisher Scientific, Cat #4368706) on a QuantStudio 6 Flex Real-Time PCR system.

### RNA-Sequencing and Analysis

RNA-sequencing libraries were prepared using 1 ng of total RNA as input. A SMART-Seq v4 Ultra Low Input RNA Kit for Sequencing (Takara) was used for cDNA synthesis, with a 10-cycle PCR enrichment. Sequencing libraries were then made using Illumina's Nextera XT Library Prep kit. The indexed libraries were sequenced on a NextSeq High-Output platform using the unpaired, 75-bp read-length sequencing protocol to obtain a total of at least 10 million reads per sample. Sequencing reads were aligned to the GRCm38/mm10 mouse reference genome using Salmon⁷⁶. Differential expression analysis was performed using the DESeq2 package⁷⁷. Statistical significance was given to genes with p-value<0.05 and absolute fold change greater than 1.5.

### Colony Formation Assay

FACS-purified HFSCs were plated on mitomycin C (P212121, Cat #M92010)-treated J2 fibroblast feeders at a density of 10,000 cells/well of 12-well plates, in E media supplemented with 15% (v/v) serum and 0.3 mM calcium as described in previous studies^{78,79}. For Gas6-treatment, E medium was supplemented with recombinant mouse Gas6 protein (R&D systems, Cat #8310-GS) at 500 ng/ml. For R428-treatment experiments, E medium was supplemented with R428 (APExBIO, Cat #A8329) at 1 μM. Cells were fixed and stained with 1% (wt/vol) Rhodamine B (Millipore Sigma, R6626). Colony diameter was measured from scanned images of plates using Image J.

### Imaging and Image Analysis

Images were obtained with a Zeiss LSM 880 confocal microscope with a 20× air objective, a 40× oil-based objective (Carl Zeiss) or a Keyence BX-700 epifluorescence microscope with ×20 or ×40 objective (Keyence). Images are presented as maximum intensity projection images or a single Z stack. Images were further processed and assembled into panels using Adobe Photoshop CC and Adobe Illustrator CC.

### Statistical Analyses

Statistical analyses were performed with GraphPad Prism 7 (GraphPad Software) with unpaired two-tailed Student's t test. The DESeq2 package utilized the Benjamini-Hochberg procedure for multiple testing correction. Genes were considered differentially expressed with an adjusted p-value below 0.05. The error bars are mean±standard error of the mean (SEM).

### Results:

### Removal of Adrenal Glands Leads to Loss of HFSC Quiescence

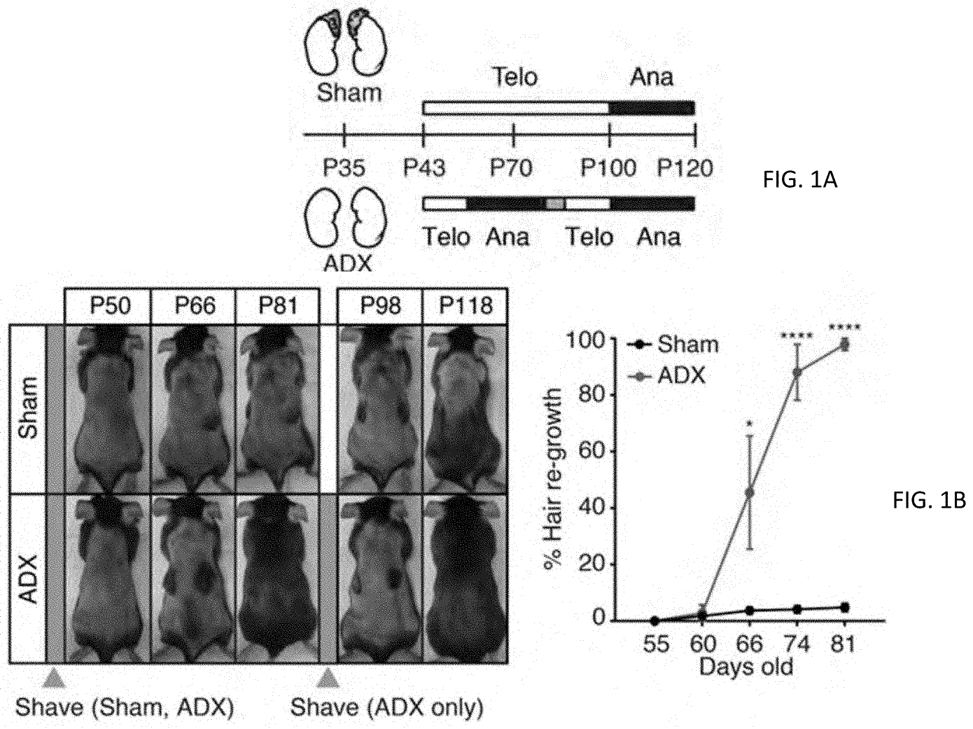

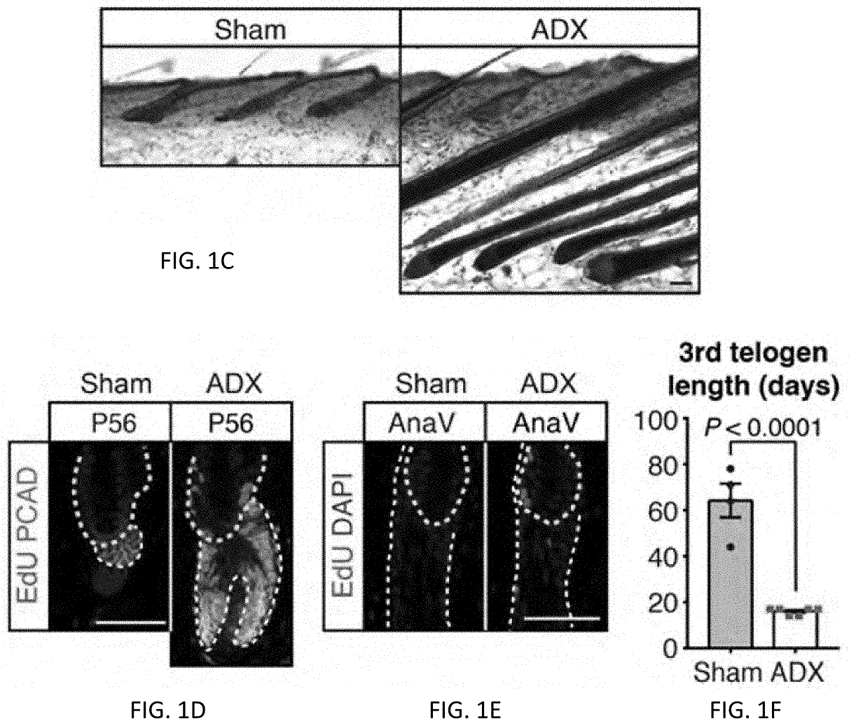

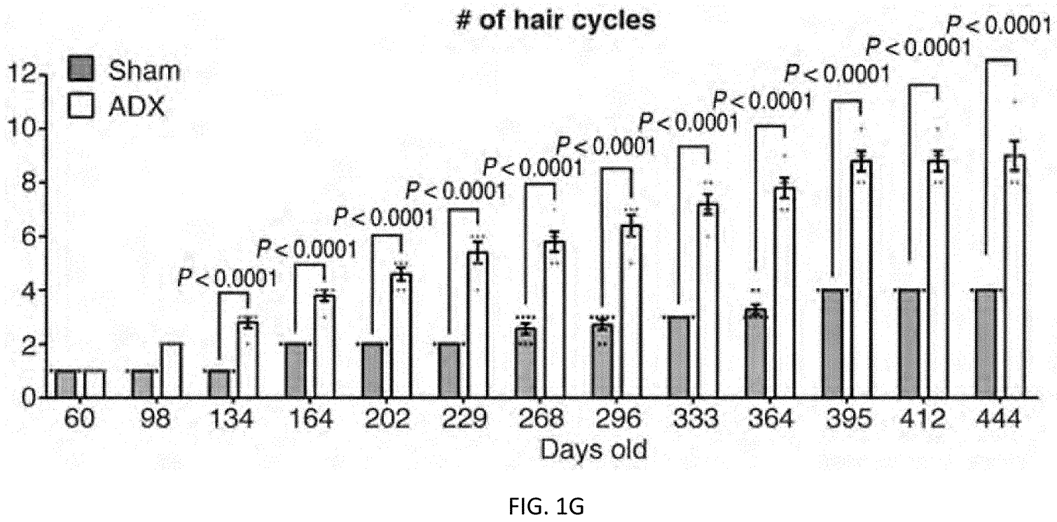

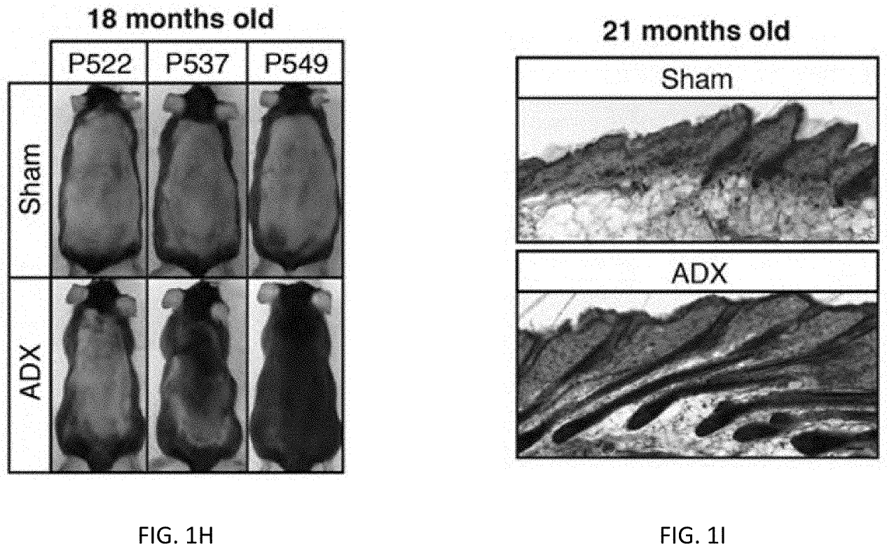

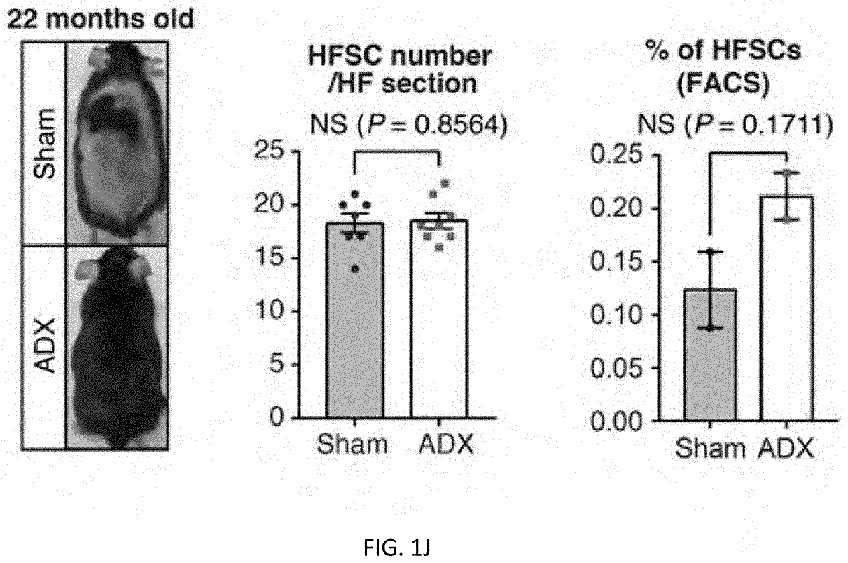

To determine if hormones from the adrenal glands impact HFSCs, both adrenal glands were surgically removed on postnatal day 35 (P35), a time before mice enter their extended second telogen (FIG. 1A). Since adrenal glands also secrete aldosterone to regulate salt balance, drinking water was supplemented with 1% w/v saline solution. Sham-operated C57BL/6 mice remained in extended telogen and began to enter anagen between P95-P110. In contrast, adrenalectomized (ADX) mice had significantly shorter telogen, and began to enter anagen around P50-P55 (FIGS. 1B-1D). Normally, HFSCs enter a brief phase of proliferation in early anagen but resume quiescence by mid-anagen¹³. In ADX mice, however, HFSCs and the upper outer root sheath region (which contributes to future HFSCs³¹) remain proliferative even in late anagen (FIG. 1E). These data suggest that HFSCs of ADX mice have lost their quiescence feature.

Loss of Quiescence does not Exhaust HFSCs Long-Term

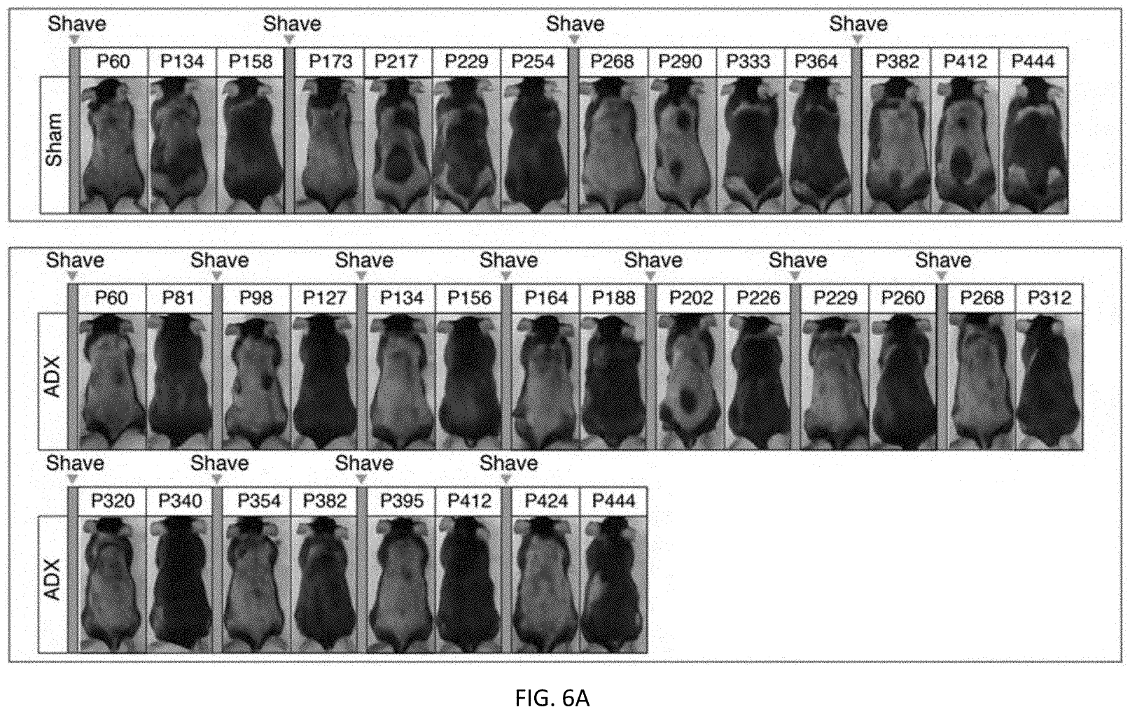

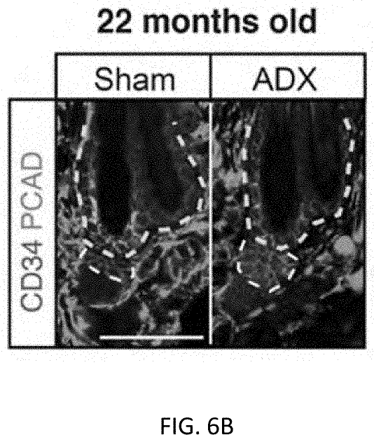

It was next asked whether ADX mice suffered consequences associated with long-term loss of quiescence in HFSCs. For this, hair cycle progression of sham-controls and ADX mice was monitored over a period of >20 months. In control animals, telogen phases following the 2nd telogen became progressively longer, and anagen entry became sporadic and asynchronized (FIGS. 1F-1G, FIG. 6A). In contrast, hair follicles in ADX mice repeatedly entered anagen in a synchronized fashion across the whole back skin (FIG. 6A). The hair follicles in ADX mice entered telogen, but only stayed in telogen for 2-3 weeks before next anagen began. Over a 15 month period following adrenalectomy, ADX mice had gone through about 9 synchronized hair cycles in their back skin. By contrast, the sham-operated mice entered anagen asynchronously, and only recovered the hair coat in their back skin 4 times (FIG. 1G). Strikingly, ADX mice displayed a normally dense hair coat as well as robust anagen entry even at 18-22 months of age (FIGS. 1H-1J). This is in sharp contrast to sham or normal old animals at the same age, in which HFSCs remain in prolonged quiescence and rarely initiate hair follicle regeneration (FIG. 1H)^{17,18,32}. H&E and immunofluorescence staining showed normal hair follicle morphology in aged ADX animals and an absence of aberrant overgrowth or hyperplasia (FIGS. 1I-1J, FIG. 6B). Moreover, HFSC numbers were maintained in spite of repeated anagen entry (FIG. 1J). These data suggest that systemic factors secreted from the adrenal glands are critical gatekeepers of telogen. These findings also indicate that in the absence of adrenal glands, the activity of HFSCs and hair follicle regeneration do not decline with age.

### HFSC Quiescence is Regulated by Adrenal Gland-Derived Corticosterone

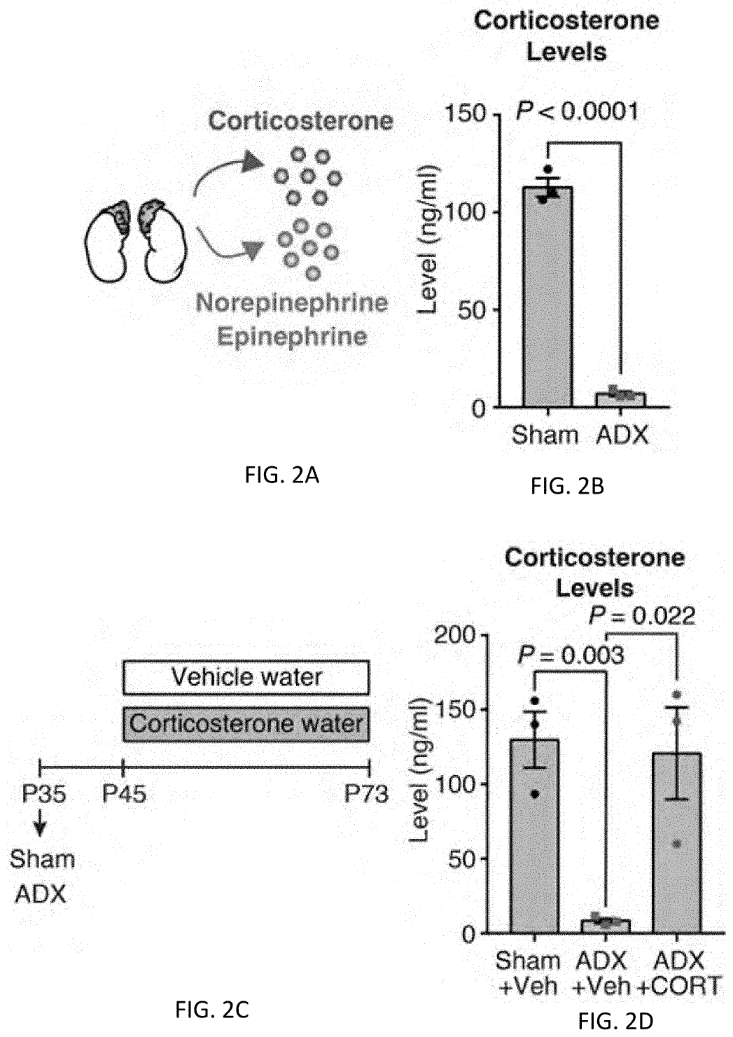

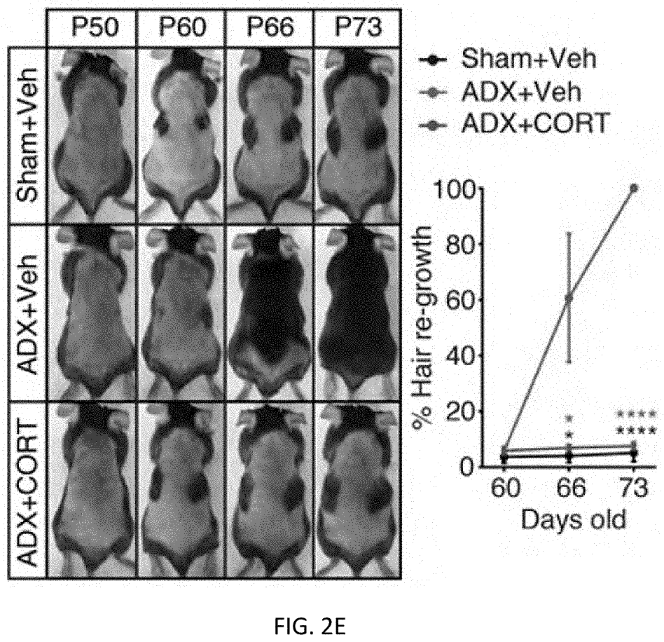

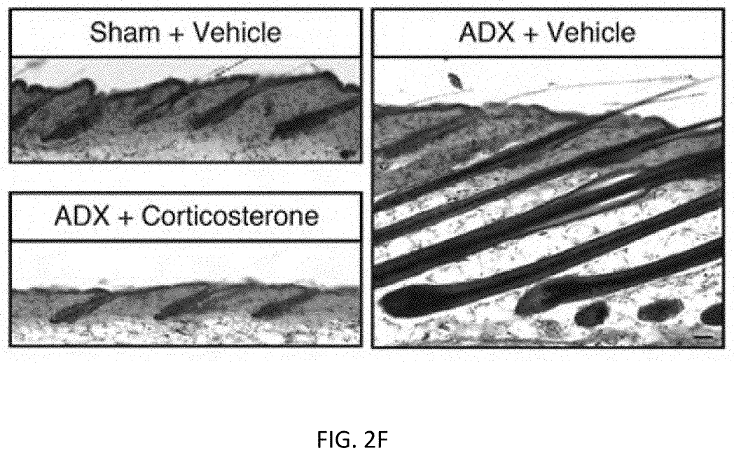

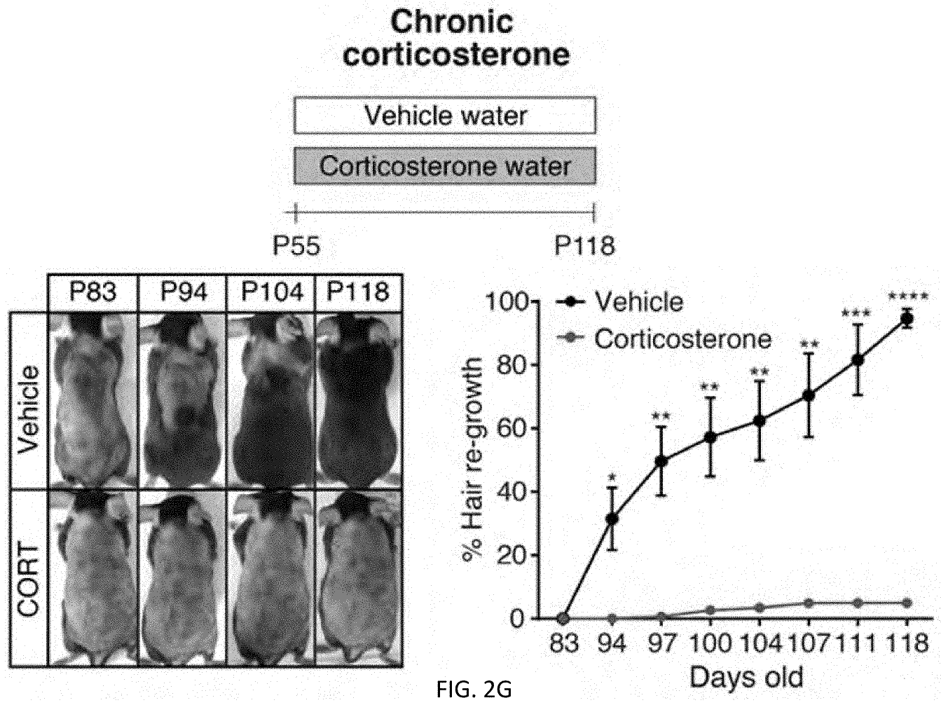

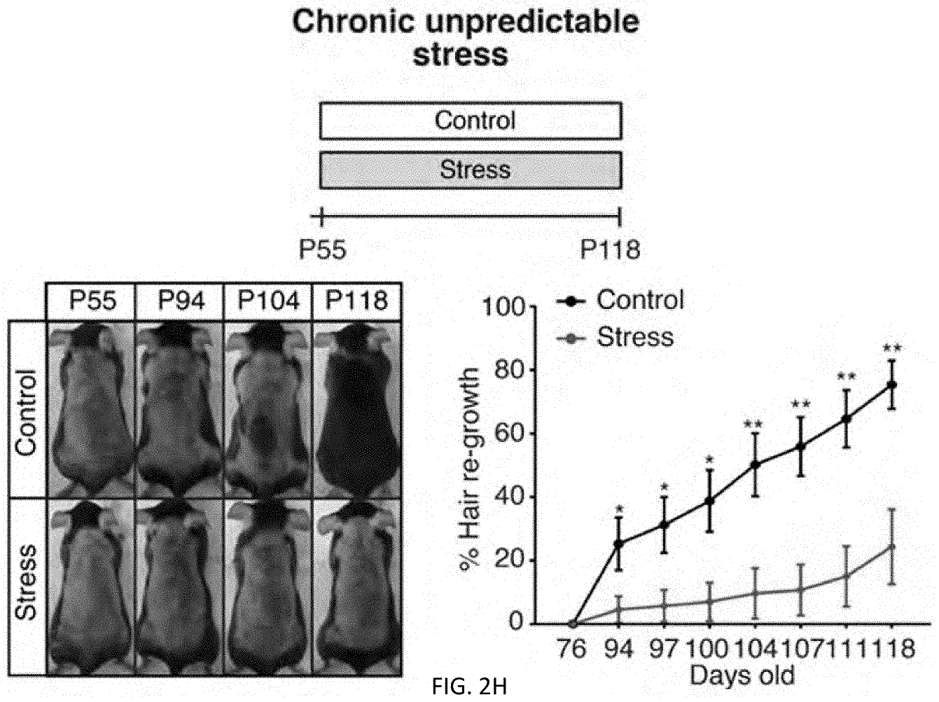

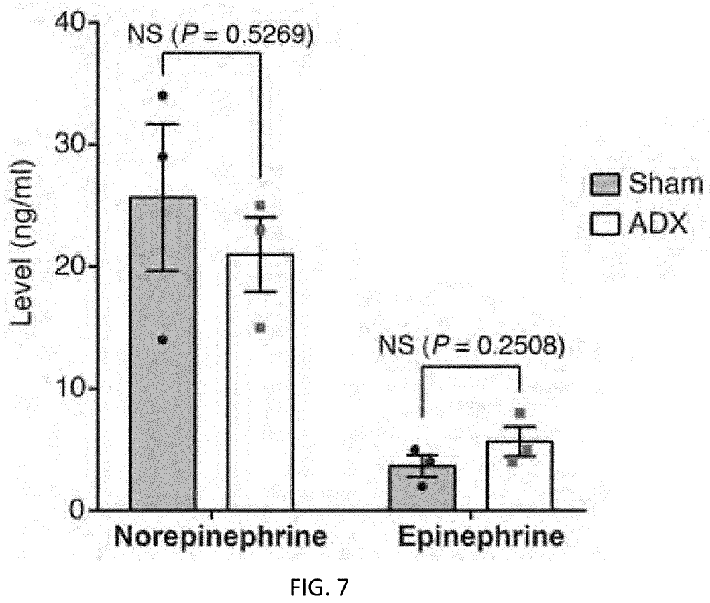

Next, the aim was to determine which adrenal gland-derived secreted factors had the capability to control HFSC quiescence. Adrenal gland produces several hormones, including corticosterone, epinephrine, norepinephrine, and aldosterone, which exert profound physiological effects in the body³³ (FIG. 2A). While aldosterone mainly functions to maintain salt balance in the kidney and colon, the other three hormones have wide ranging effects across organ systems in normal or stress conditions³⁴. To identify which hormone(s) controls HFSC quiescence, levels of each hormone were quantified in control and ADX animals. Levels of epinephrine and norepinephrine were not significantly altered following adrenalectomy under steady state, but levels of corticosterone dropped substantially, and became barely detectable (FIG. 2B, FIG. 7). These data suggest that after adrenalectomy, the major hormonal change in the circulation is a reduction in the stress hormone corticosterone.

It was next determined if lack of corticosterone was a primary reason for ADX animals to enter precocious anagen. To this end, corticosterone levels in ADX animals were restored by the addition of corticosterone to their drinking water (FIGS. 2C-2D). It was found that supplementation of corticosterone effectively suppressed the aberrant activation of HFSCs we had observed in the ADX mice (FIGS. 2E-2F). Altogether, these results suggest that corticosterone secreted from the adrenal glands acts as a key systemic regulator to maintain HFSC quiescence. The data also identify corticosterone as a primary enforcer of telogen phases under normal physiological conditions.

### Elevated Corticosterone Levels Inhibit HFSC Activation

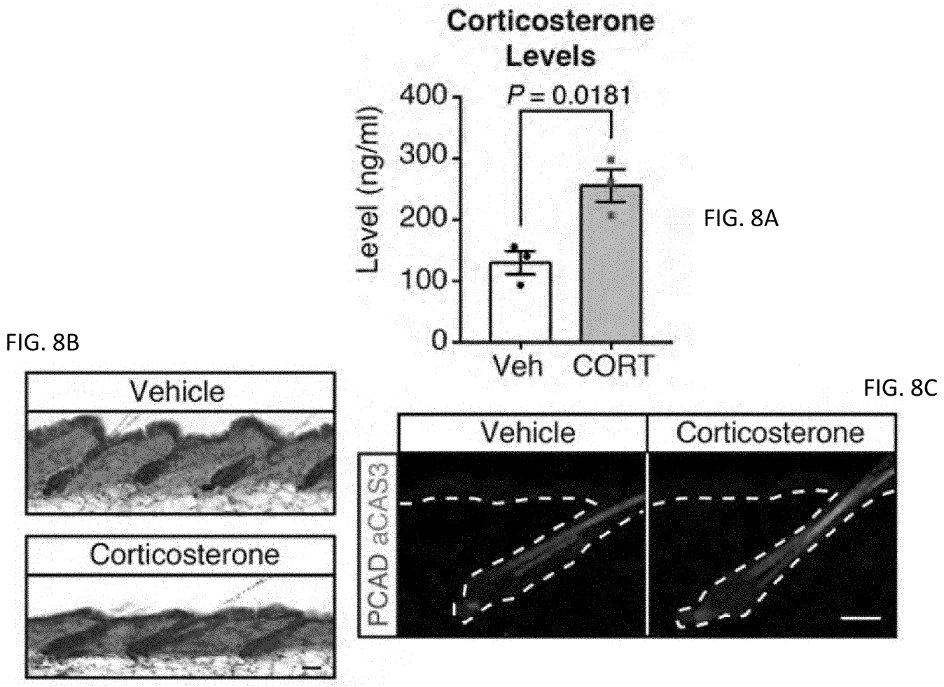

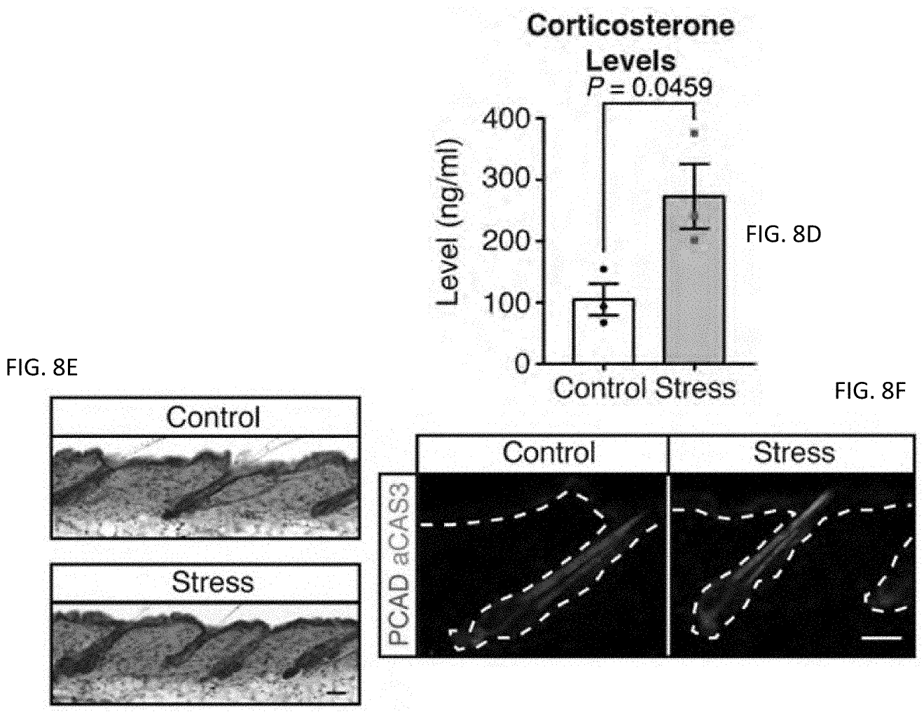

It was next asked if elevated corticosterone levels inhibited HFSC activation. For this, wild-type mice were given supplemental corticosterone in their drinking water in the second telogen, which led to enhanced corticosterone levels in the circulation (FIG. 2G, FIG. 8A). While vehicle-treated mice entered anagen around P90-P100, corticosterone-treated mice stayed in telogen, and continued to remain in telogen as long as the mice received supplemental corticosterone (FIG. 2G, FIG. 8B). Despite a significant delay in anagen entry, the hair follicle does not display abnormal cell death (FIG. 8C). These findings suggest that corticosterone is a potent suppressor of anagen entry, and that corticosterone is both necessary and sufficient to maintain telogen.

### Chronic Stress Affects Anagen Entry Through Up-Regulating Corticosterone

Next it was evaluated if chronic stress impacts hair follicle regeneration and HFSC activity, and if stress exerts its effect through corticosterone. For this, a chronic unpredictable stress model widely used in behavioral neuroscience was adapted, in which different mild stress stimuli are given to mice daily and in an unpredictable manner^{35,36}. As expected, the stressed mice displayed elevated corticosterone levels (FIG. 8D). Similar to corticosterone-treatment, HFSCs of mice subjected to chronic unpredictable stress displayed significantly extend telogen and a substantial delay in anagen entry (FIG. 2H, FIGS. 8E-8F).

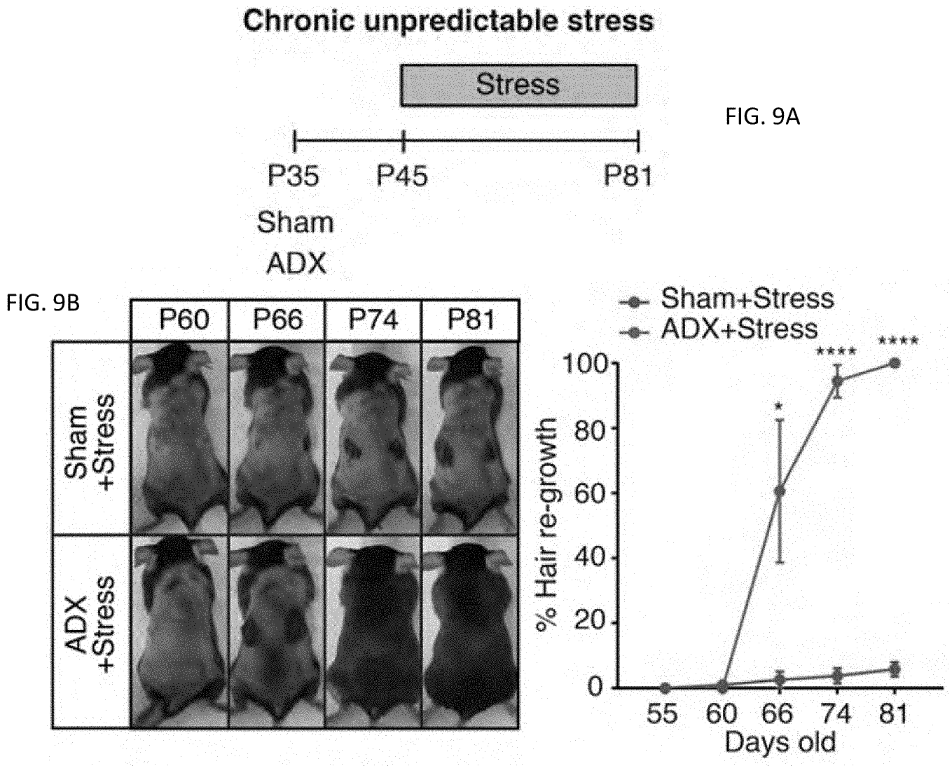

To assess if adrenal gland-derived corticosterone mediates the impact of stress on HFSCs, ADX and sham mice were subjected to chronic unpredictable stress. The ADX animals displayed significantly shortened telogen and accelerated anagen entry under stress, suggesting that hormones from the adrenal glands are essential in mediating the inhibitory effects of stress on HFSC activity (FIGS. 9A-9B). Collectively, the data shows that elevated corticosterone levels, whether stress-induced or provided exogenously, cause HFSCs to become refractory to activation, resulting in significantly extended telogen.

### Corticosterone Acts on the Dermal Niche to Regulate HFSC Quiescence

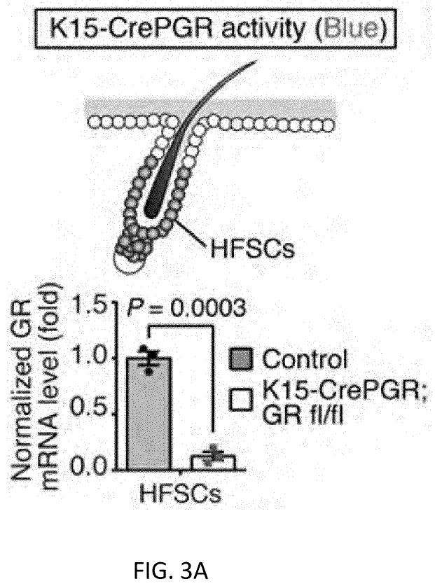

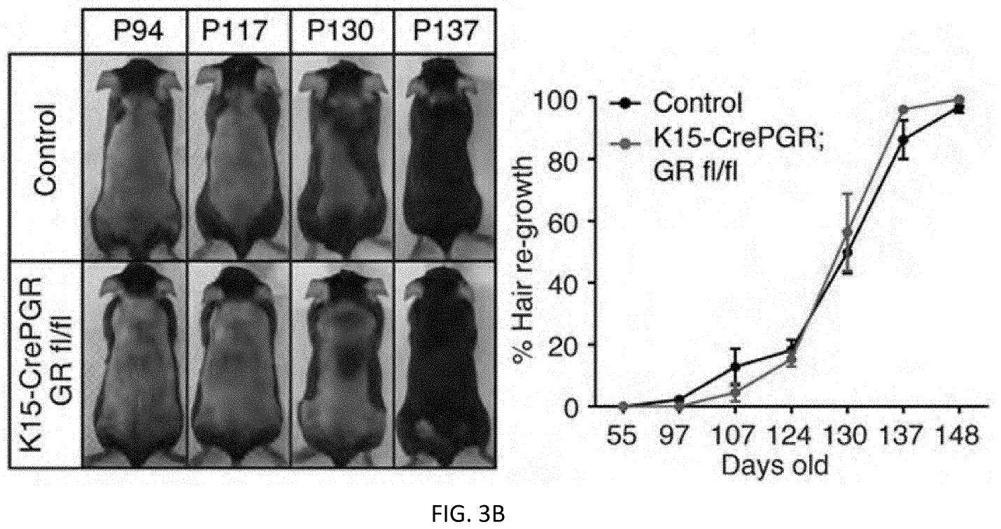

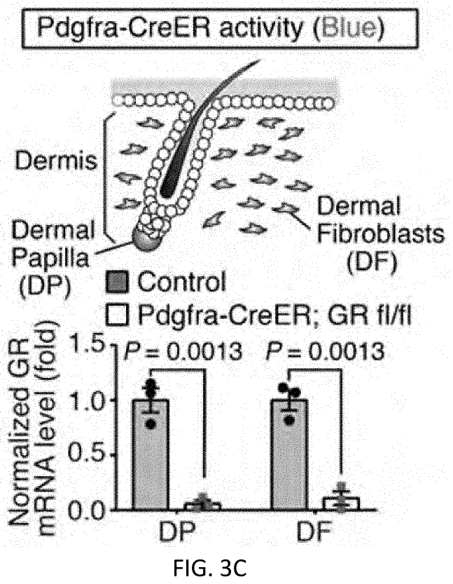

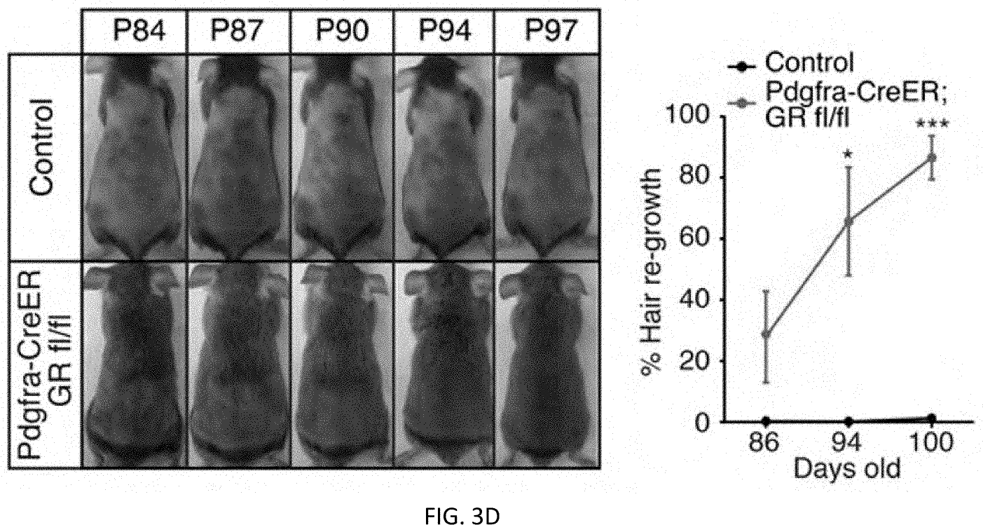

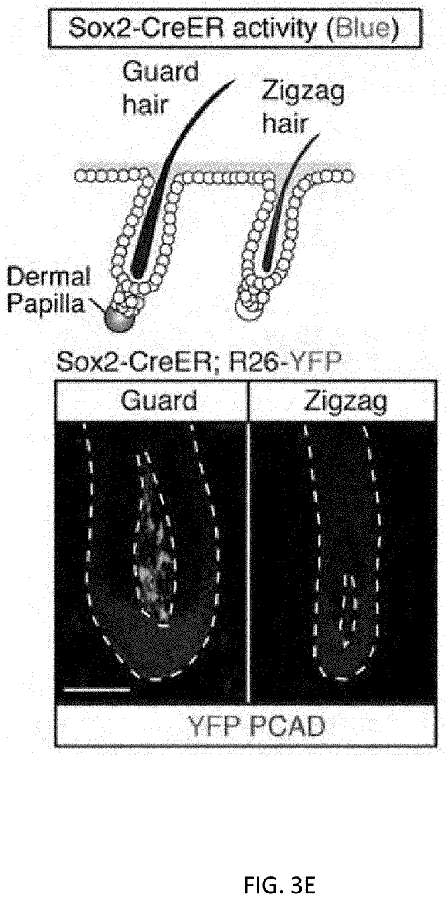

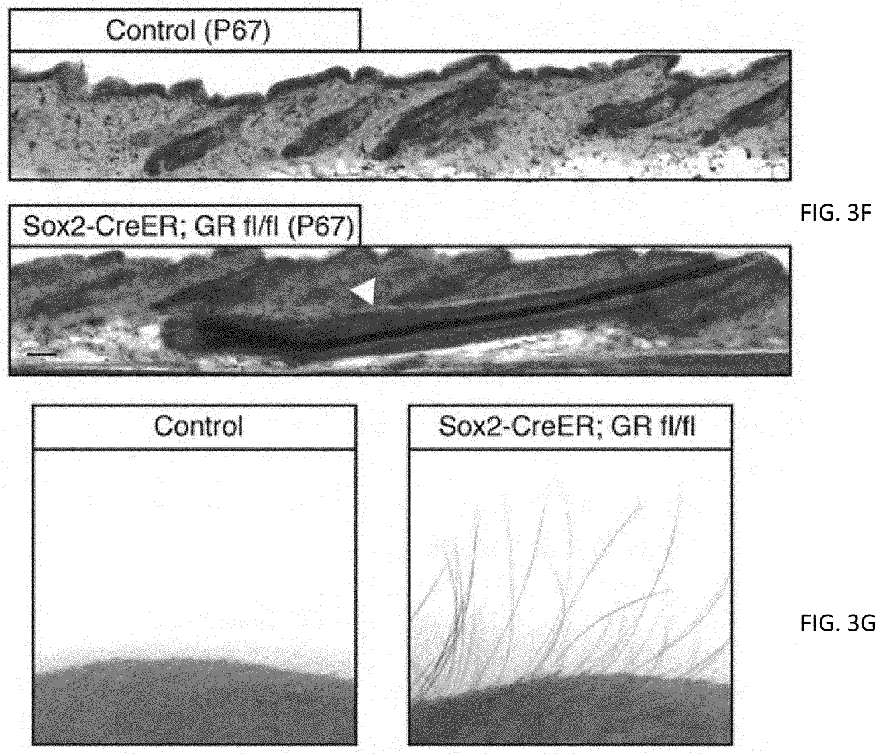

Next, the aim was to identify which cell types corticosterone acts on to regulate HFSC quiescence. First it was asked if corticosterone regulates HFSCs directly. To this end, the receptor for corticosterone was depleted—glucocorticoid receptor (GR)³⁷—from HFSCs using K15-CrePGR. Despite efficient depletion of GR in HFSCs, the K15-CrePGR; GRfl/fl mice did not display significant differences in telogen length or HFSC activity (FIGS. 3A-3B), indicating that corticosterone does not act on HFSCs directly to regulate HFSC quiescence.

It was then asked if corticosterone acts on cells within the HFSC niche to influence stem cell activity. Dermal fibroblasts are a diverse and heterogenous population surrounding the HFSCs. Some dermal fibroblast subpopulations, including dermal papilla (DP) cells and adipocyte precursor cells, are known to regulate HFSC activity³⁸⁻⁴⁰. To test the possibility that corticosterone regulates HFSC activity via the dermal niche, we depleted GR using Pdgfra-CreER, a driver expressed in the majority of fibroblast populations including DP and adipocyte precursor cells⁴¹. qRT-PCR analysis confirmed that GR was efficiently knocked out in the dermis (FIG. 3C). Similar to ADX animals, Pdgfra-CreER; GRfl/fl mice displayed significantly shorter telogen length and precocious anagen entry, suggesting that dermal fibroblasts mediate the effects of corticosterone on HFSC activity (FIG. 3D).

To determine if corticosterone acts predominantly through specific dermal fibroblast subsets, GR was deleted using Sox2-CreER, a driver expressed in a subset of DP cells42. Sox2-CreER; Rosa-YFP analysis suggested that the CreER is mostly active in the DP of guard hairs and absent in the other hair follicle types (FIG. 3E). Sox2-CreER; GRfl/fl animals displayed precocious anagen entry in guard hair follicles, but not in other hair follicles where Sox2-CreER activity was not detected (FIGS. 3F-3G). Altogether, these results indicate that corticosterone acts primarily on the DP to promote telogen.

### Identification of Dermal Niche Factors Regulated by Corticosterone

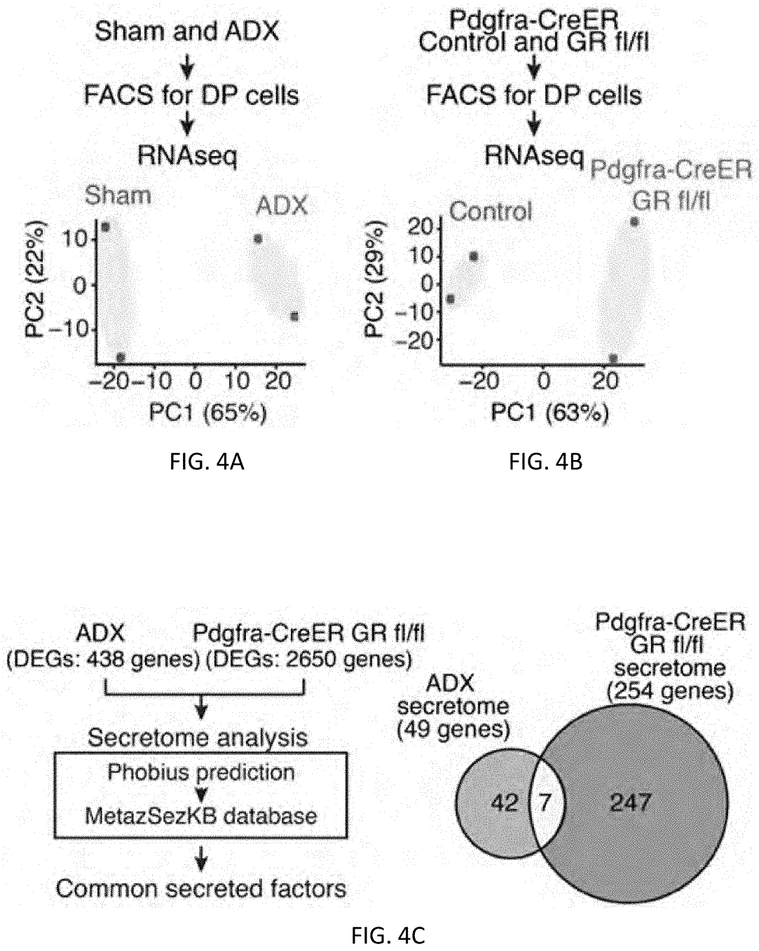

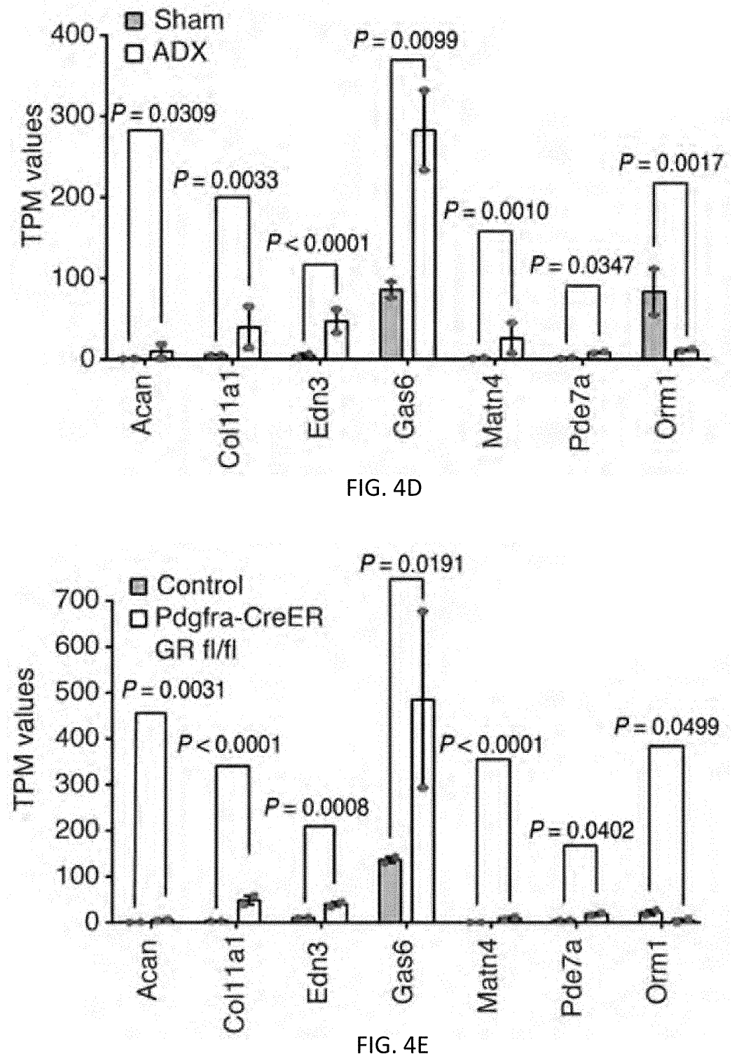

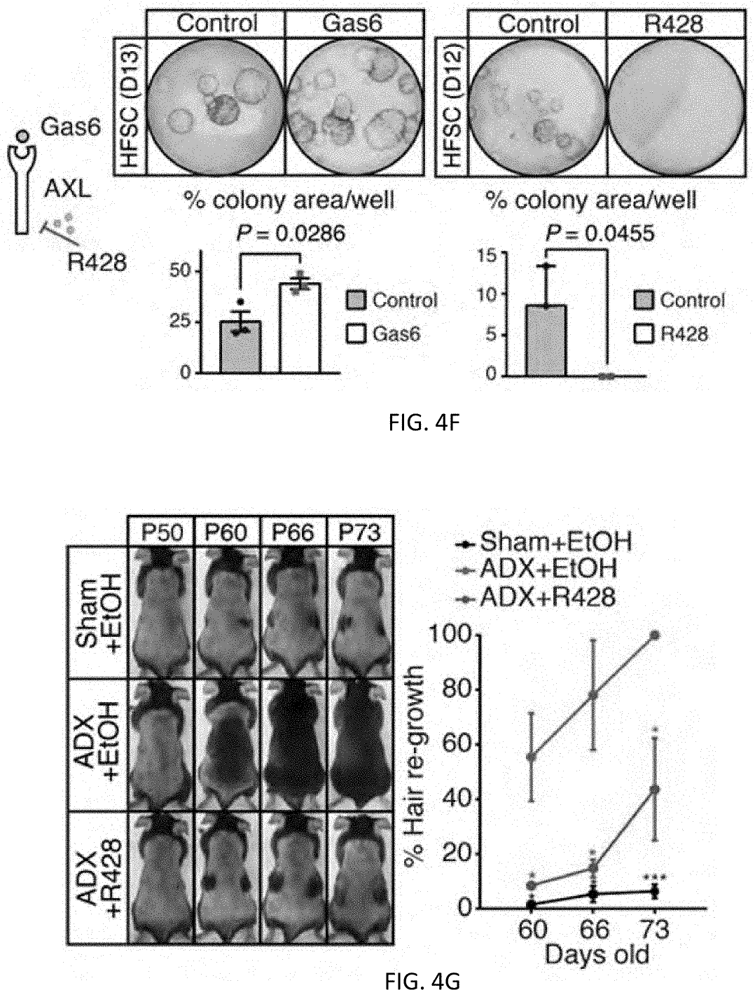

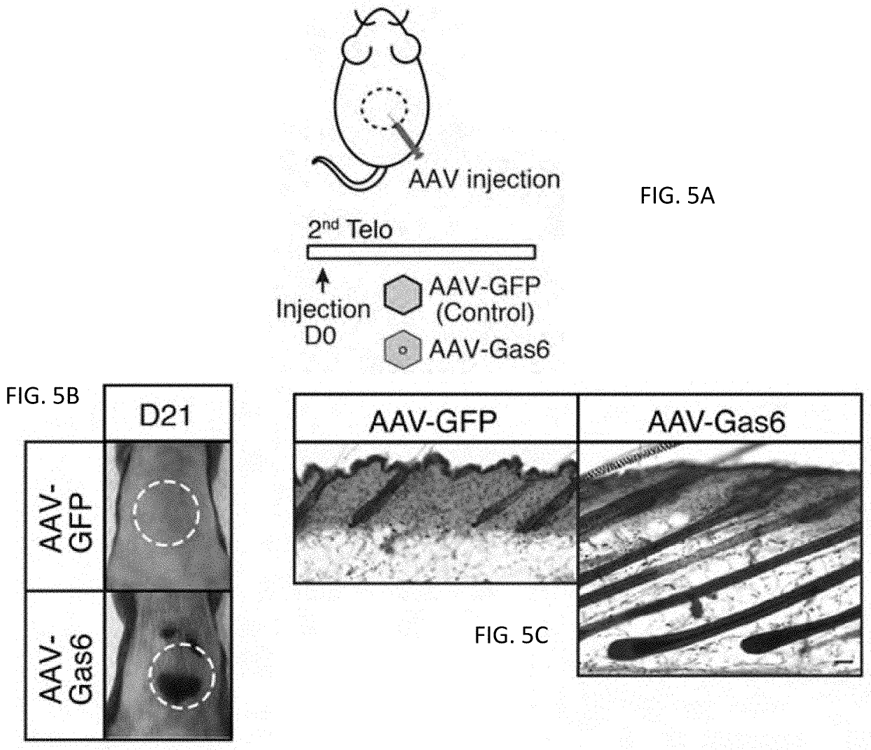

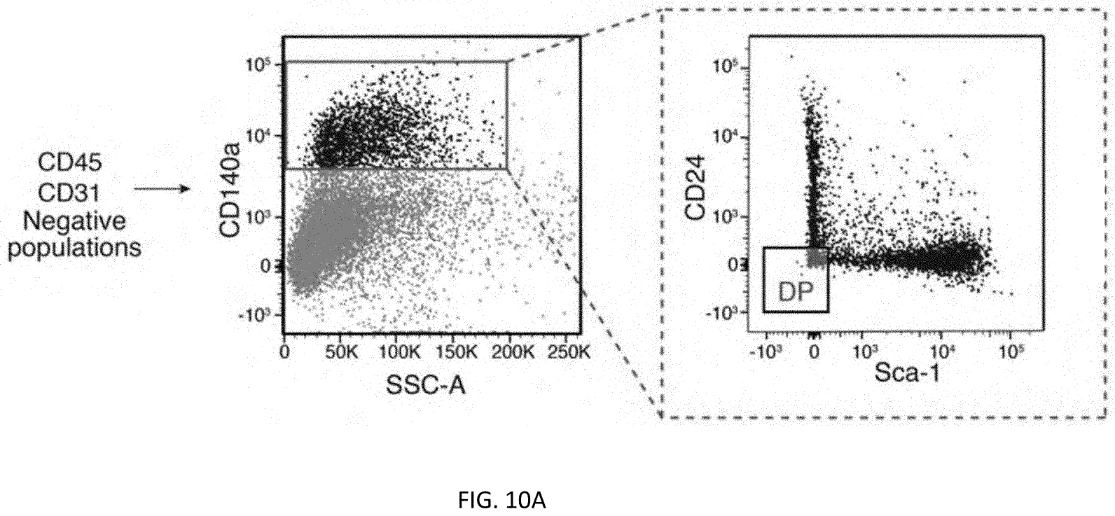

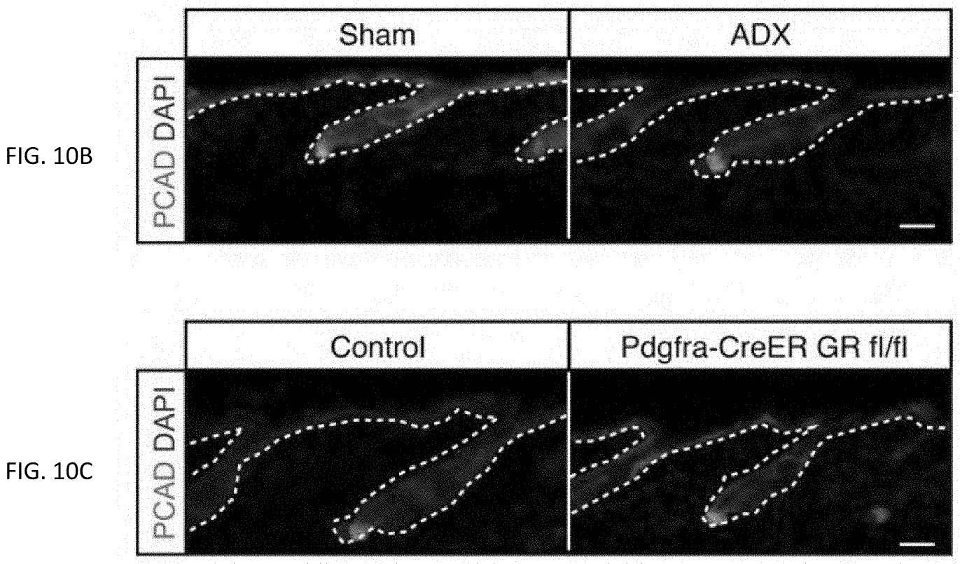

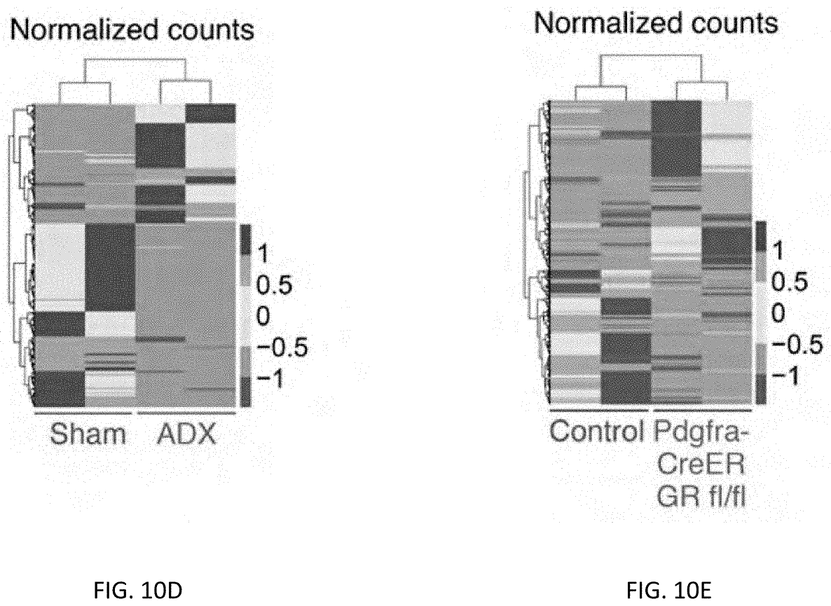

Next the aim was to identify specific dermal factors under the control of corticosterone. To this goal, RNAseq was used to analyze FACS-enriched DP cells from sham and ADX animals. In parallel, we also conducted RNAseq of DP from control and Pdgfra-CreER; GRfl/fl animals (FIGS. 4A-4B). To avoid changes simply tracked with different hair cycle stages, RNAseq analysis of DP cells was conducted isolated by flow cytometry when all samples were at telogen (FIGS. 10A-10C).

Since DP cells likely exert regulatory effects on HFSCs via secreted proteins, comparative secretome analysis was conducted to identify differentially expressed secreted factors in ADX or dermal GR-knockout DP cells. For this, differentially expressed genes (1.5-fold, P<0.05) in DP were first identified upon adrenalectomy or dermal GR depletion (FIGS. 4A4B, FIGS. 10D-10E). Then shared secreted factors were identified using two secretome databases (Phobius and MetazSecKB). This approach identified a total of 7 shared secreted factors whose expression levels are significantly altered in the DP of ADX animals or Pdgfra-CreER; GRfl/fl animals (FIG. 4C). Among the candidate secreted factors, Gas6 stood out as being abundantly expressed and significantly up-regulated in the DP of both ADX and GR KO mice (FIGS. 4D-4E). Gas6 encodes a gamma-carboxyglutamic acid-containing secreted protein43, and is a known ligand for the TYRO3, AXL, and MERTK (TAM) family of receptor tyrosine kinases, but predominantly binds to AXL⁴⁴.

### Gas6-AXL Pathway Relays the Effect of Corticosterone to HFSCs

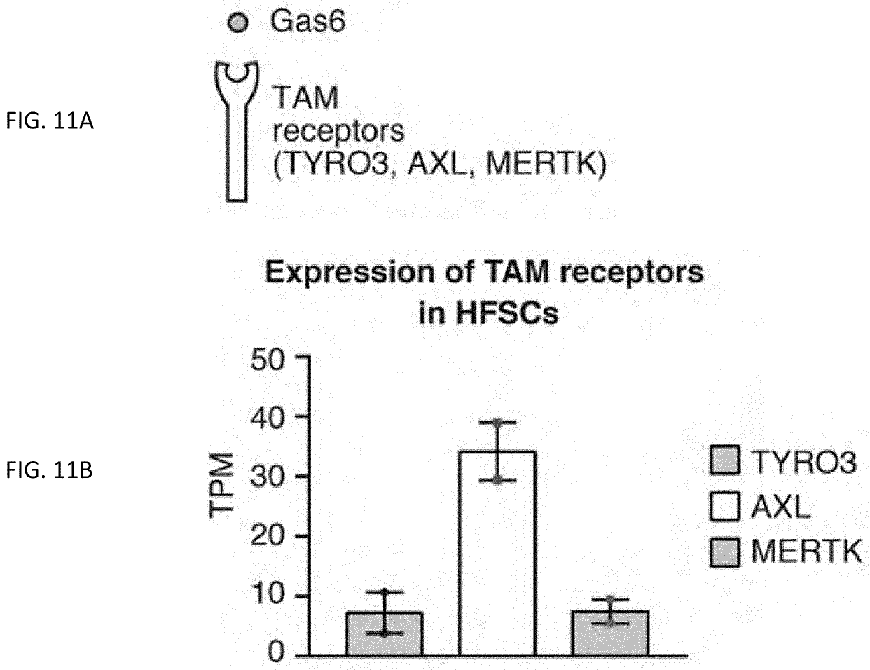

To examine the function of Gas6 in HFSCs, both gain-of-function and loss-of-function strategies were employed. It was first determined if addition of Gas6 promoted growth of HFSCs in culture. To this end, HFSCs were FACS-purified, plated them in culture, and added recombinant Gas6 proteins into the media. HFSCs formed more colonies in the presence of Gas6, suggesting that Gas6 indeed promotes HFSC growth (FIG. 4F). Notably, of all the TAM receptors, AXL is the most highly expressed in HFSCs (FIGS. 11A-11B). Indeed, blocking AXL activity with the AXL-specific inhibitor R428⁴⁵ inhibited HFSC colony formation in vitro (FIG. 4F). Moreover, topical application of R428 onto the back skins of ADX mice suppressed their precocious anagen entry, suggesting that blocking Gas6-AXL pathway can in part suppresses the aberrant HFSC activity seen with loss of corticosterone (FIG. 4G).

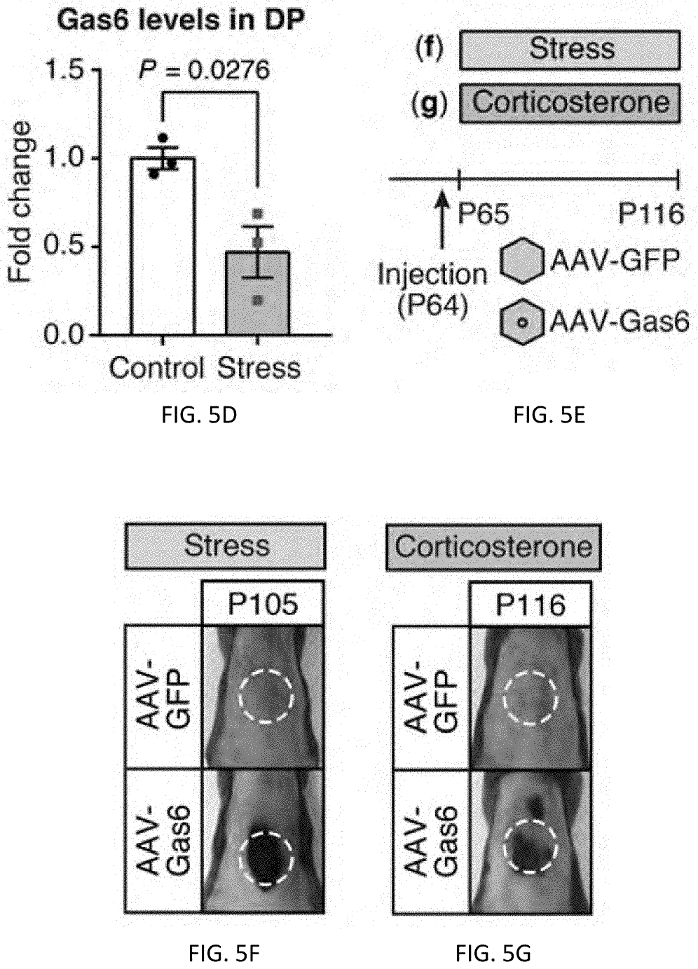

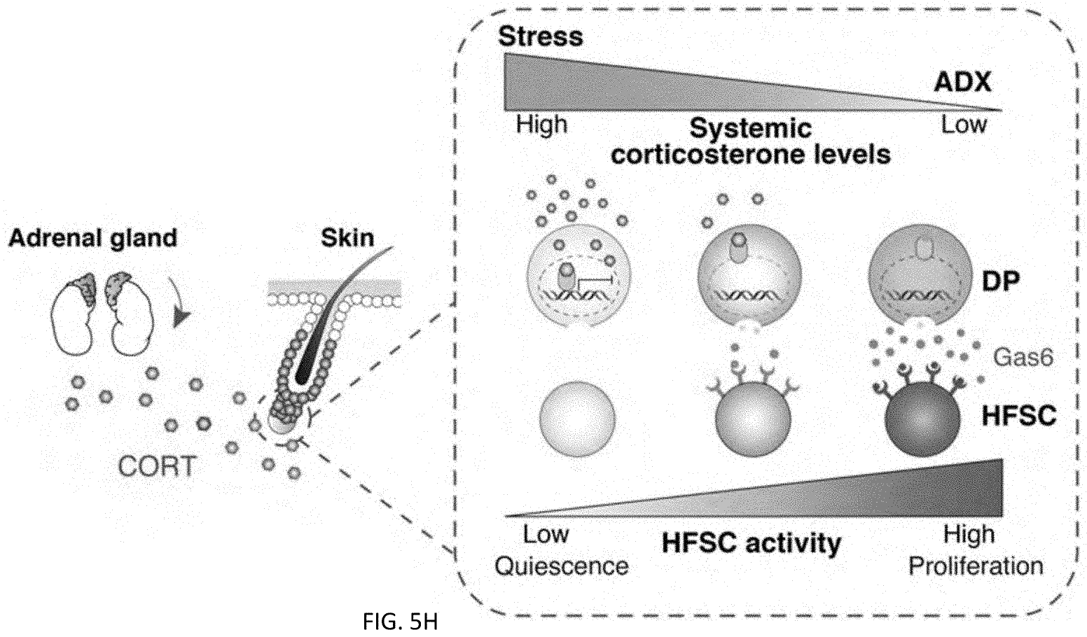

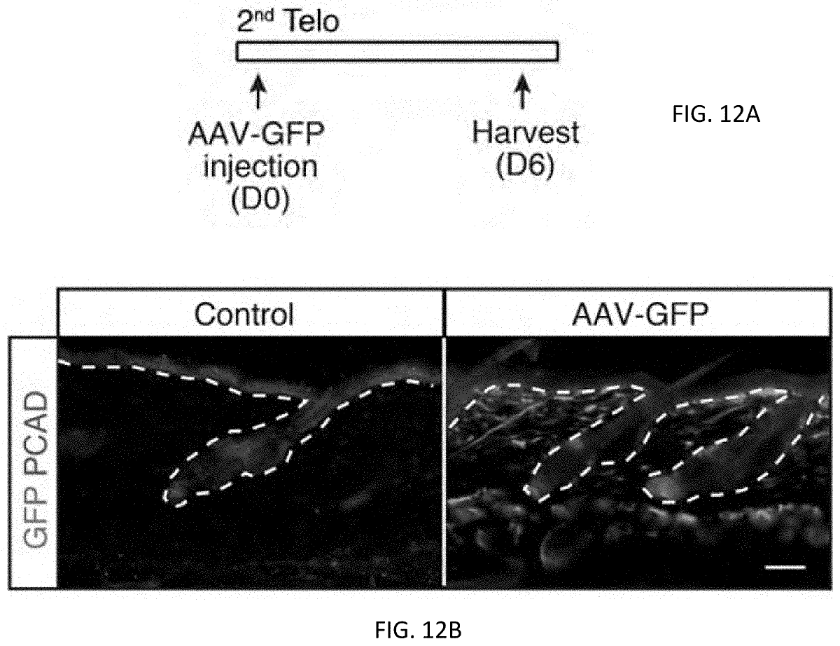

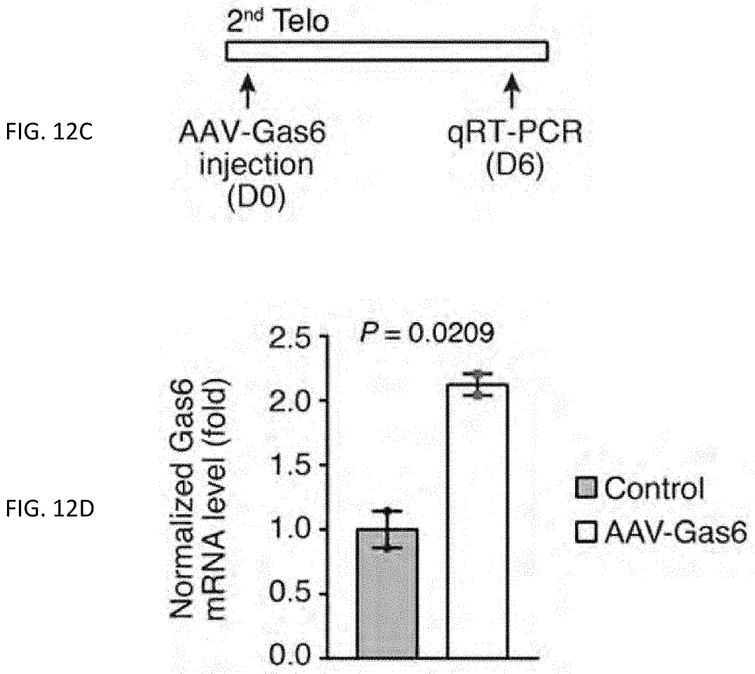

To further evaluate the potency of Gas6 in promoting HFSC activation, a construct expressing Gas6 was generated under control of the constitutively expressed CAG promoter. CAG-GFP and CAG-Gas6 were packaged into adeno-associated virus (AAVs)⁴⁶, and injected these AAVs into the skin through intradermal injections (FIG. 5A, FIGS. 12A-12B). qRT-PCR confirmed that Gas6 transcripts were up-regulated in the dermal fibroblasts within the AAV injected regions (FIGS. 12C-12D). Strikingly, AAV-CAG-Gas6 injection promoted precocious hair cycle entry at the site of injection (FIGS. 5B-5C).

### Gas6 Promotes HFSC Activation and Hair Follicle Regeneration Under Chronic Stress

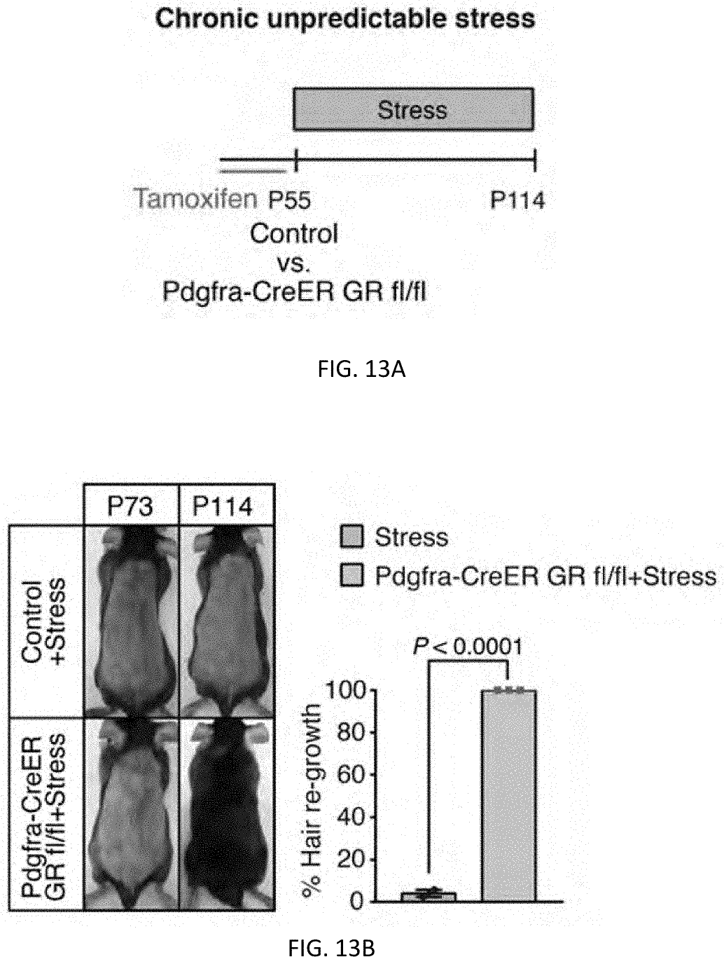

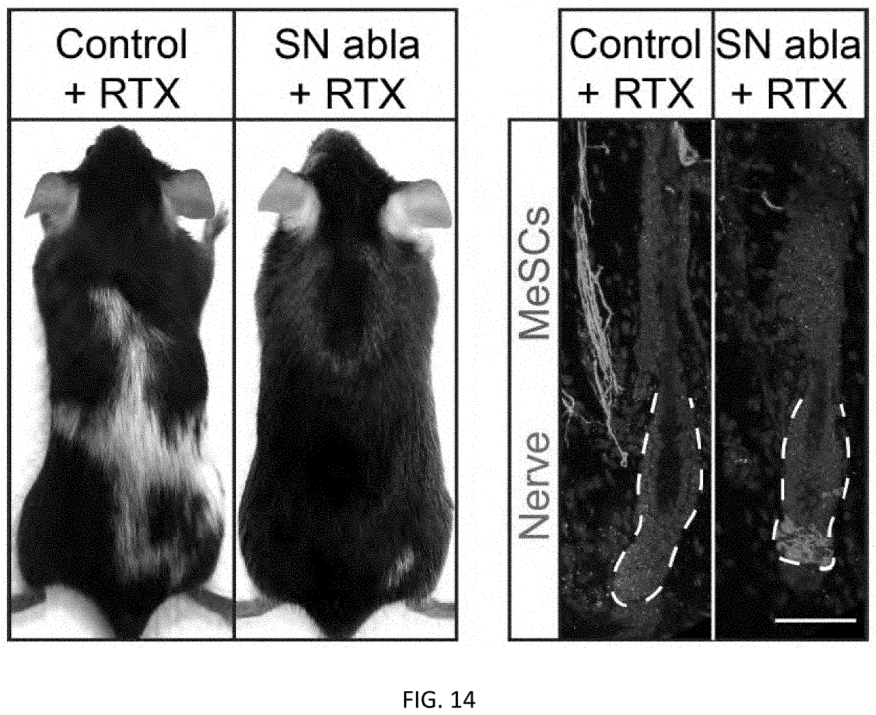

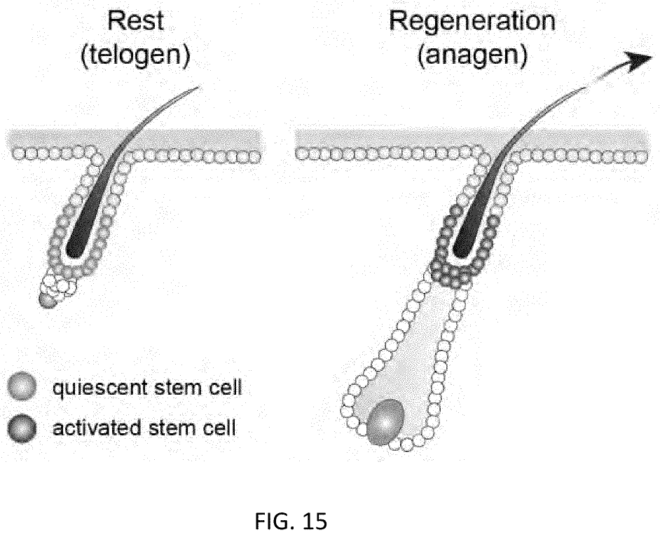

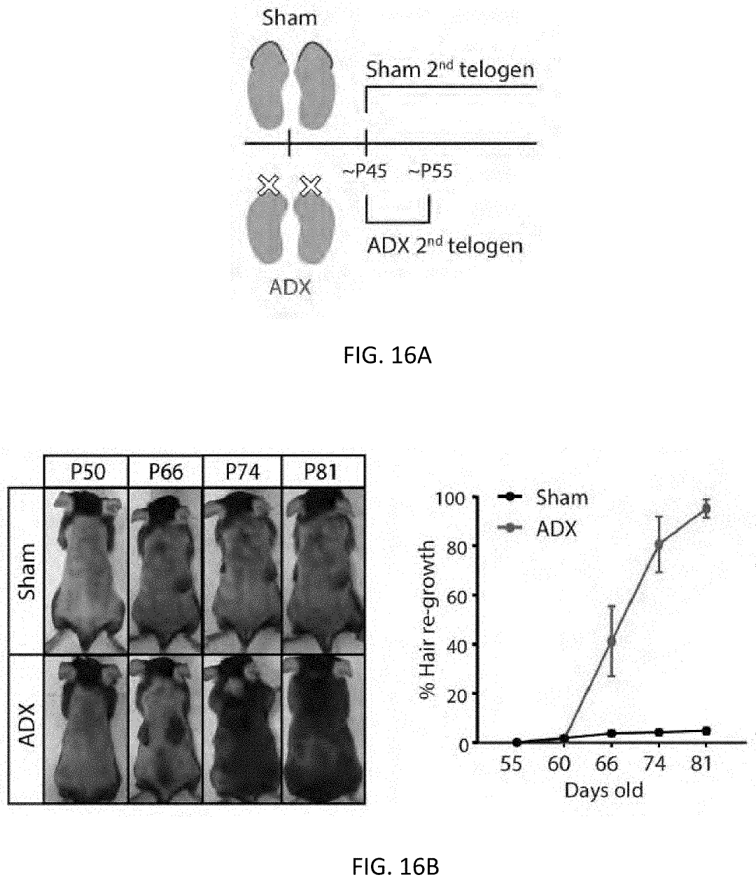

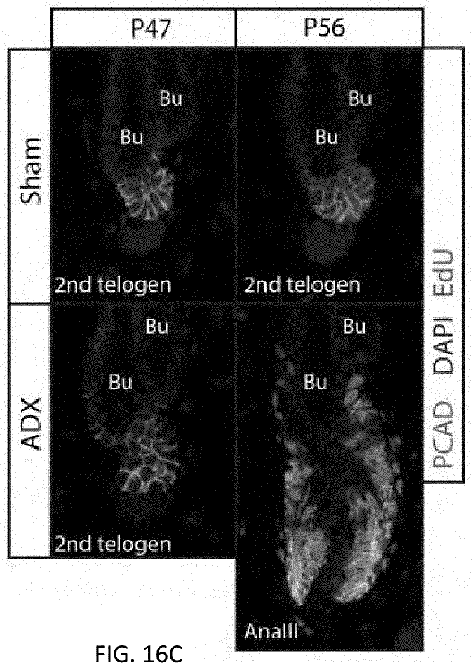

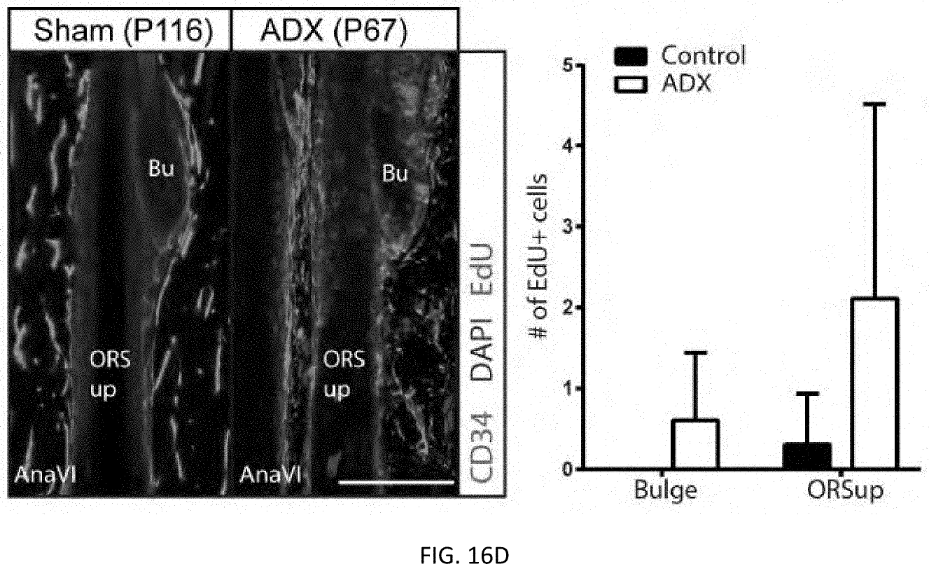

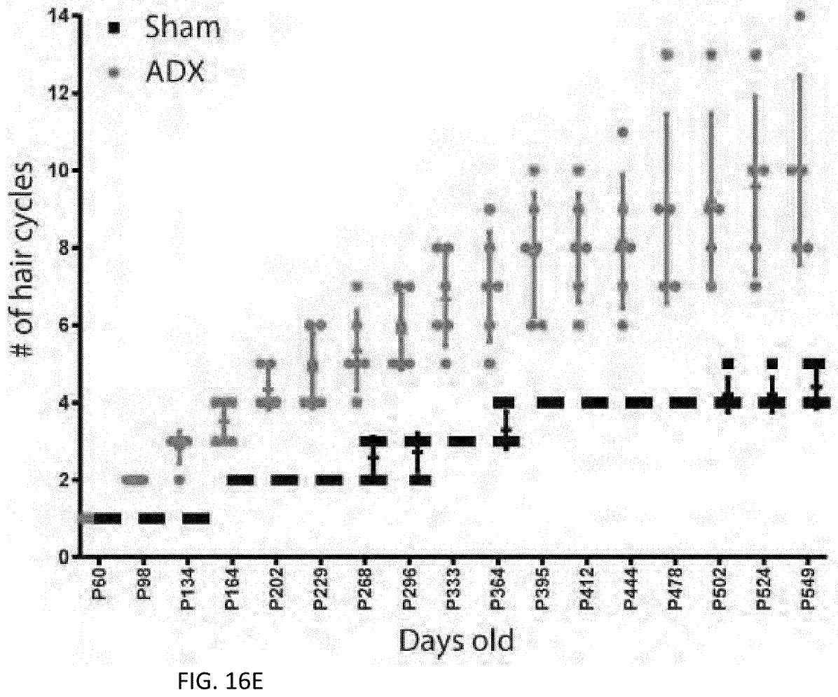

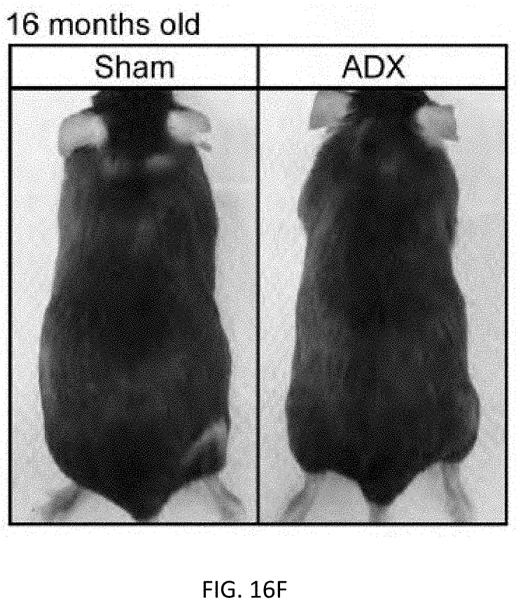

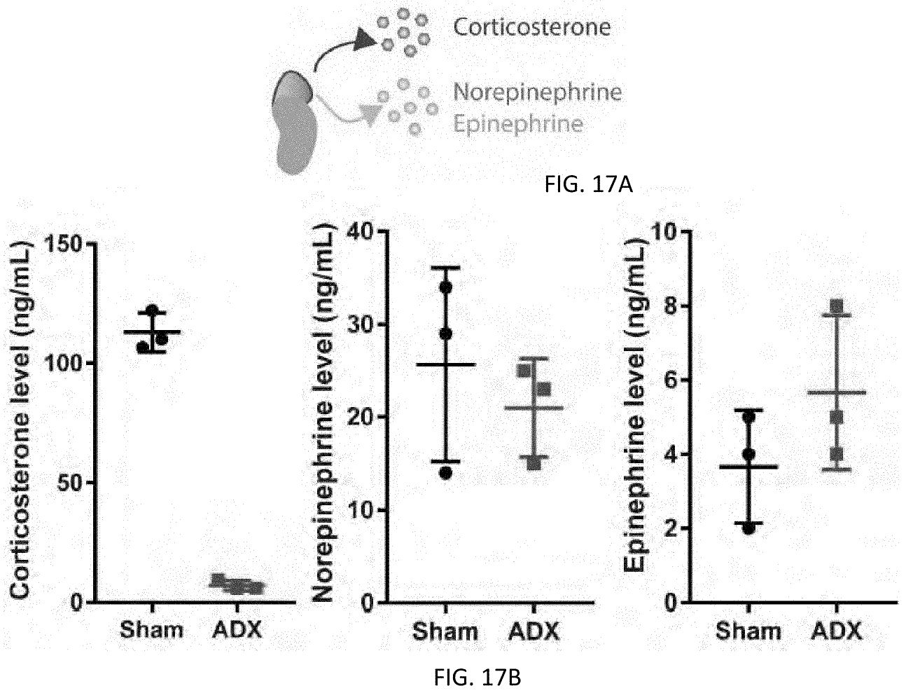

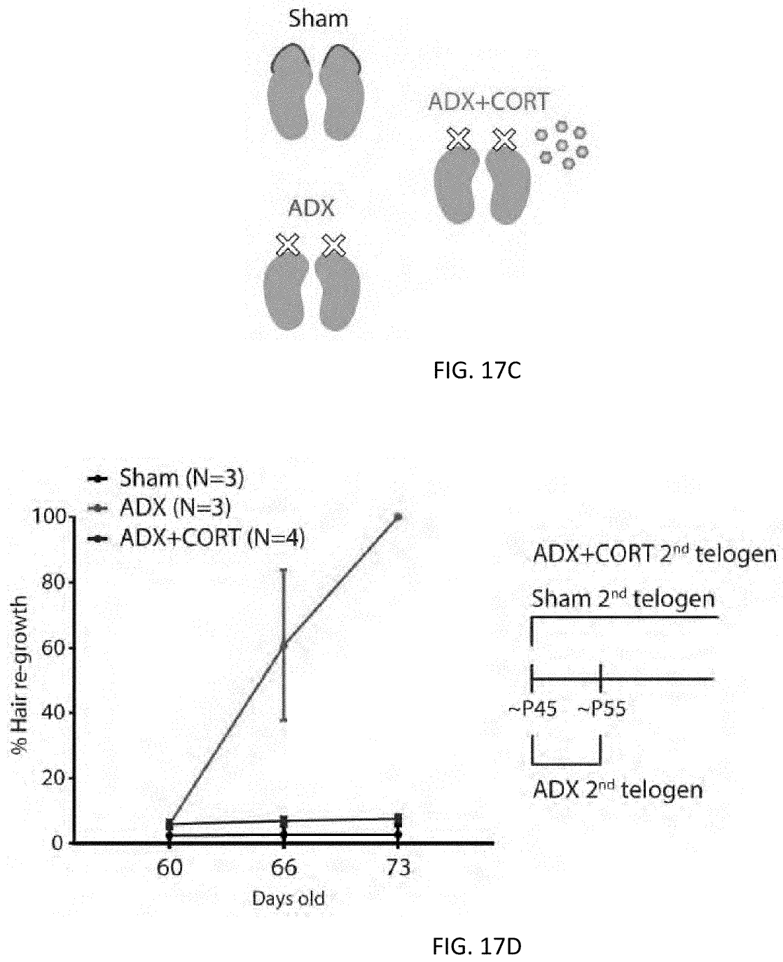

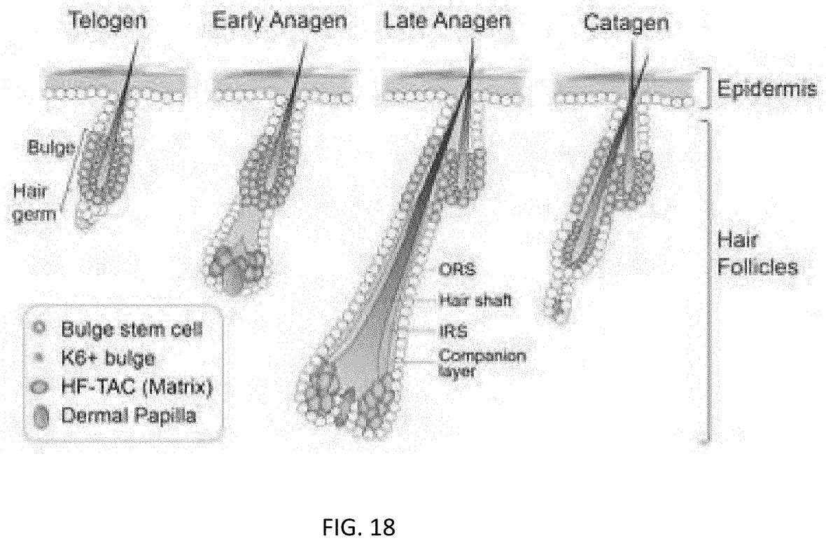

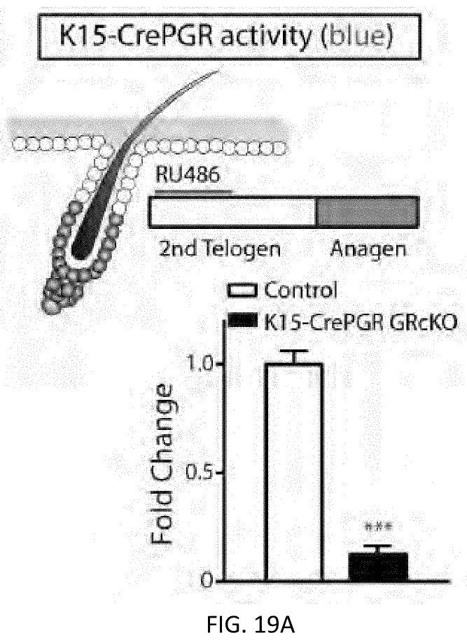

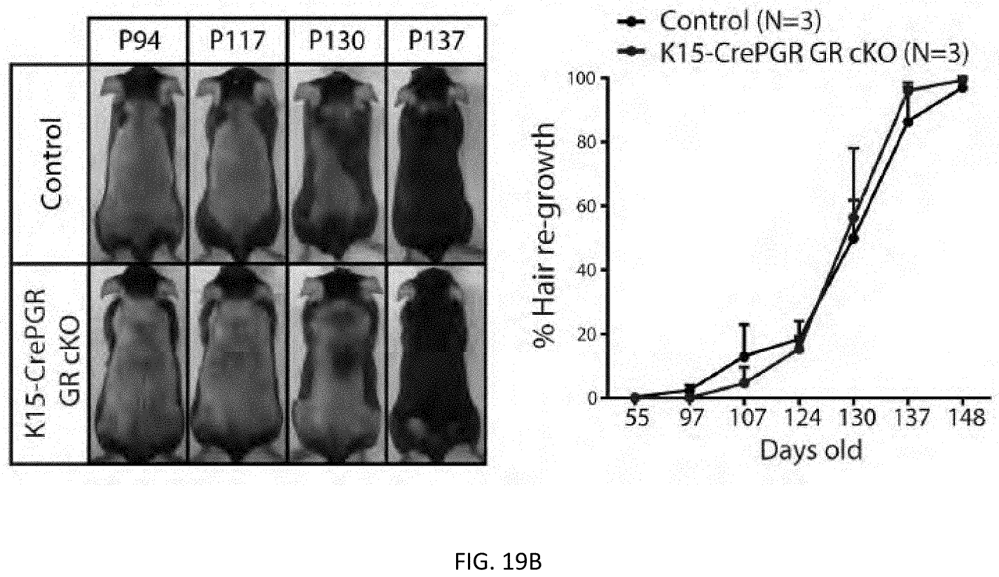

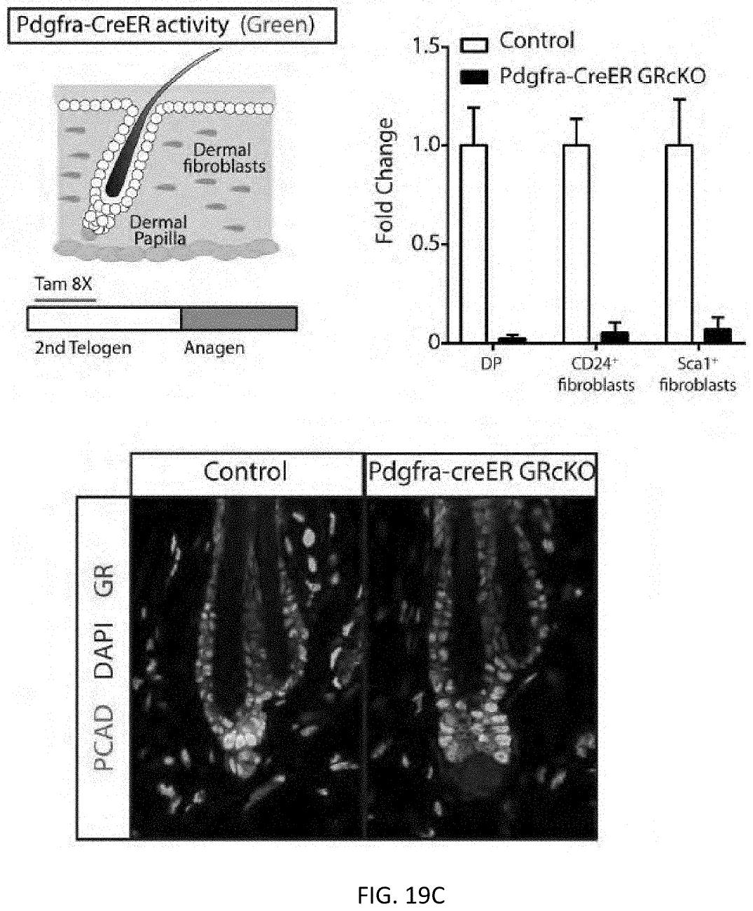

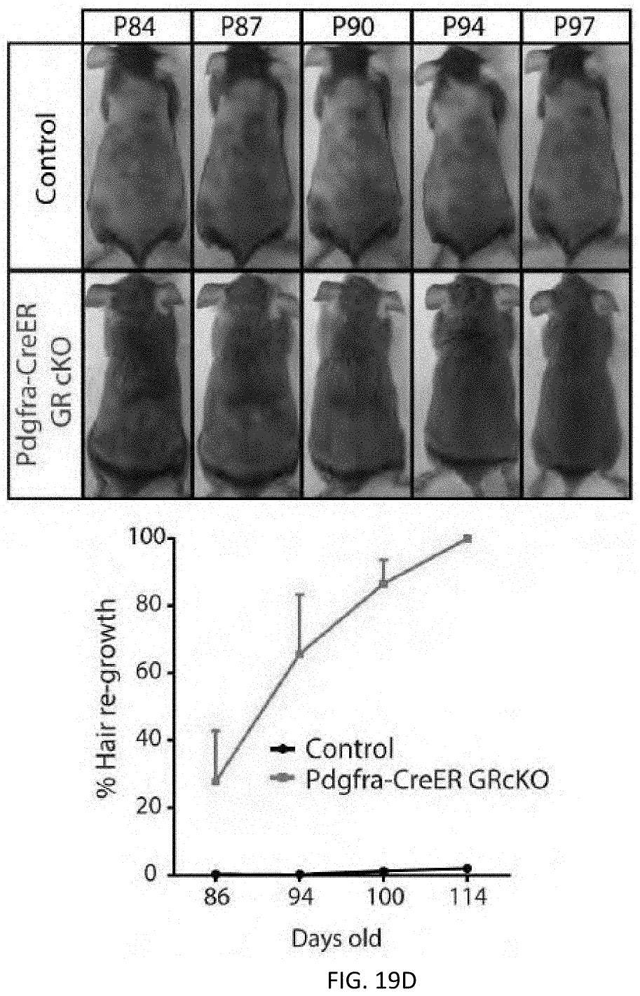

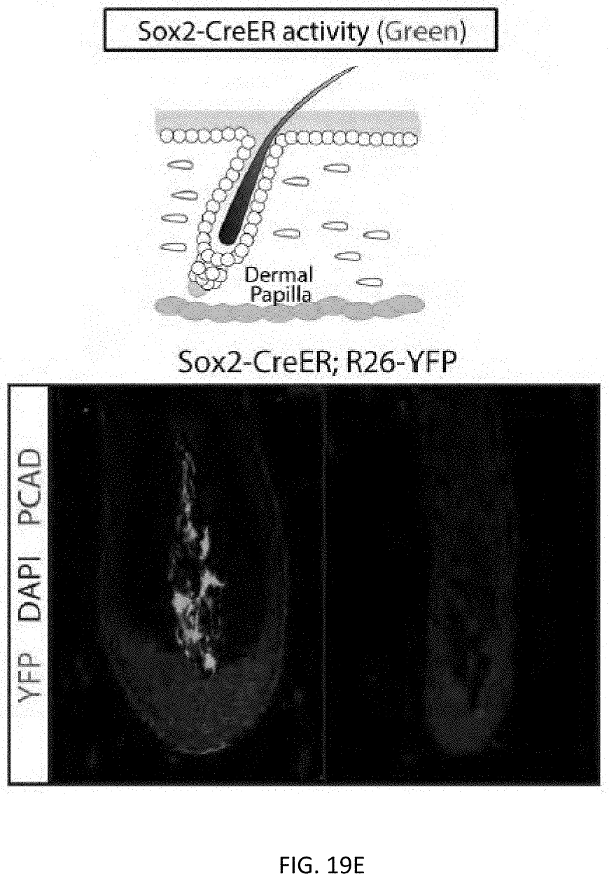

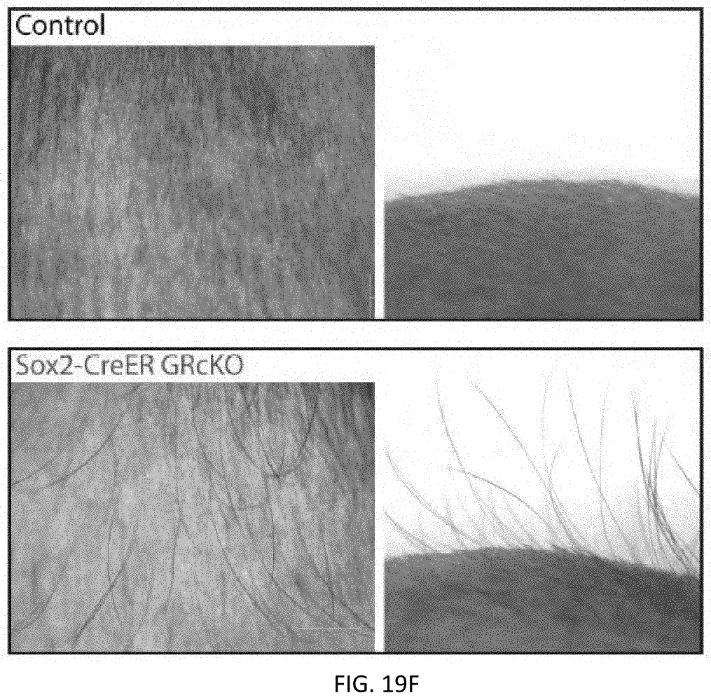

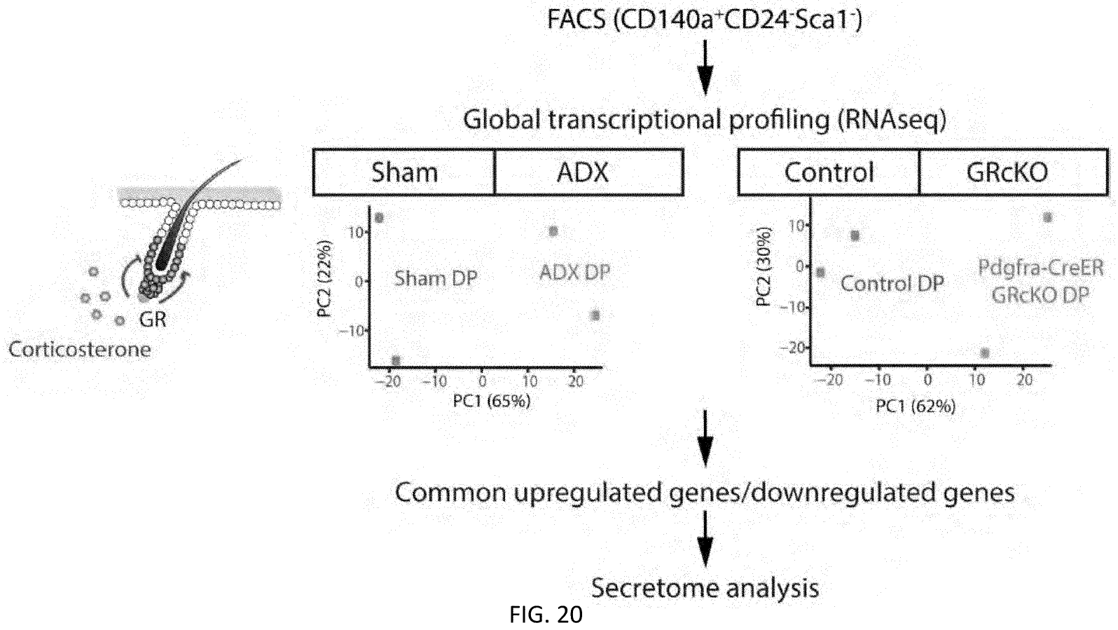

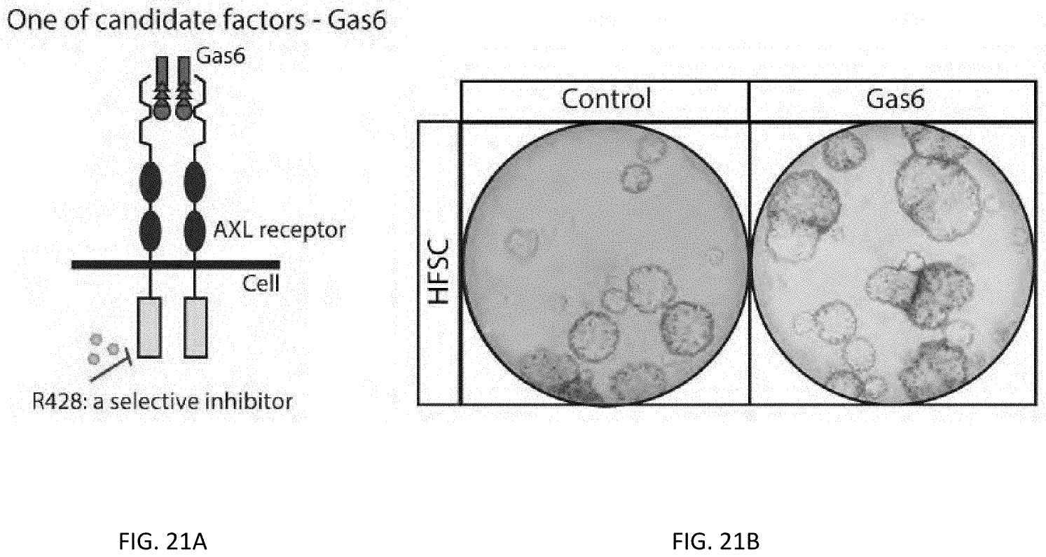

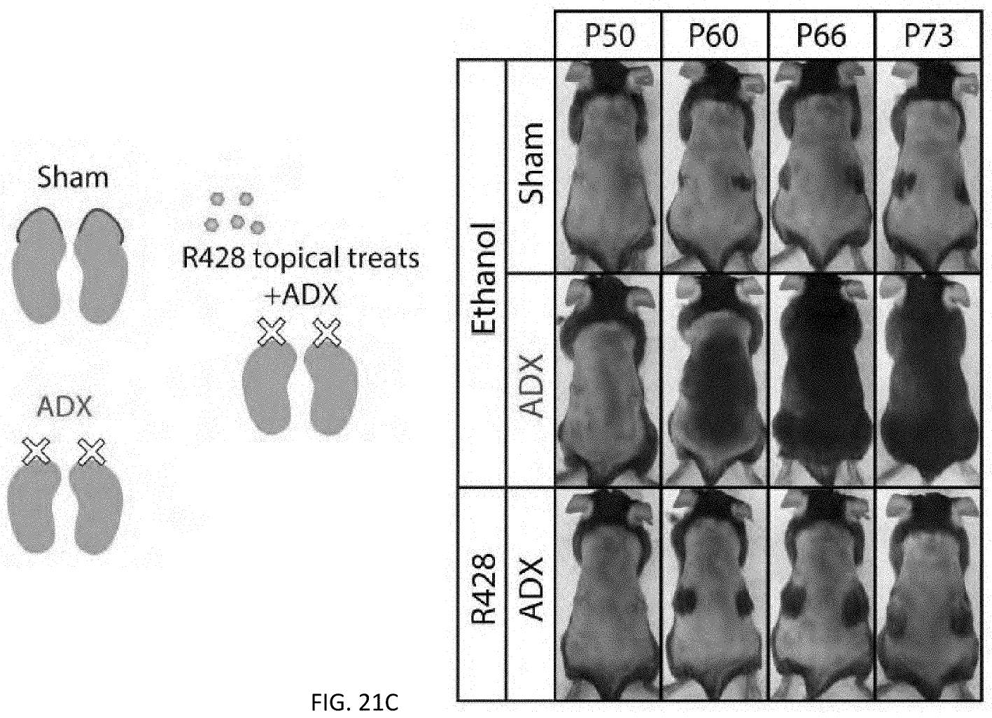

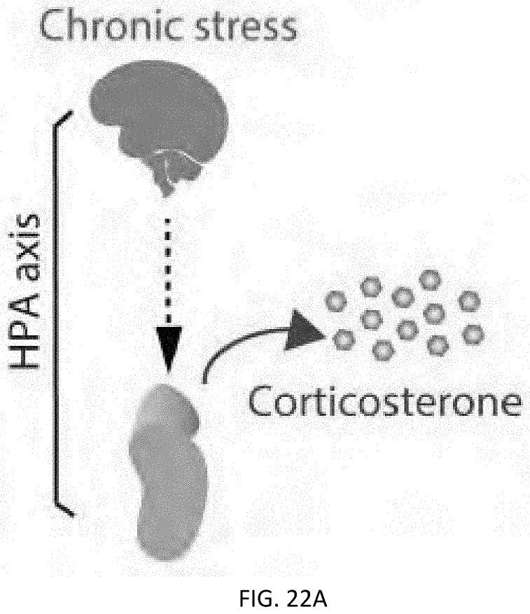

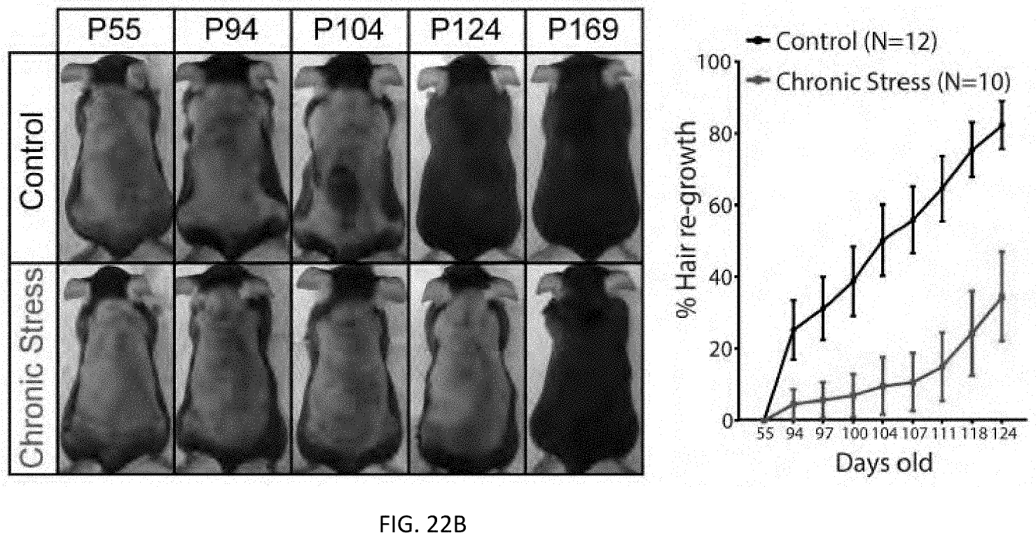

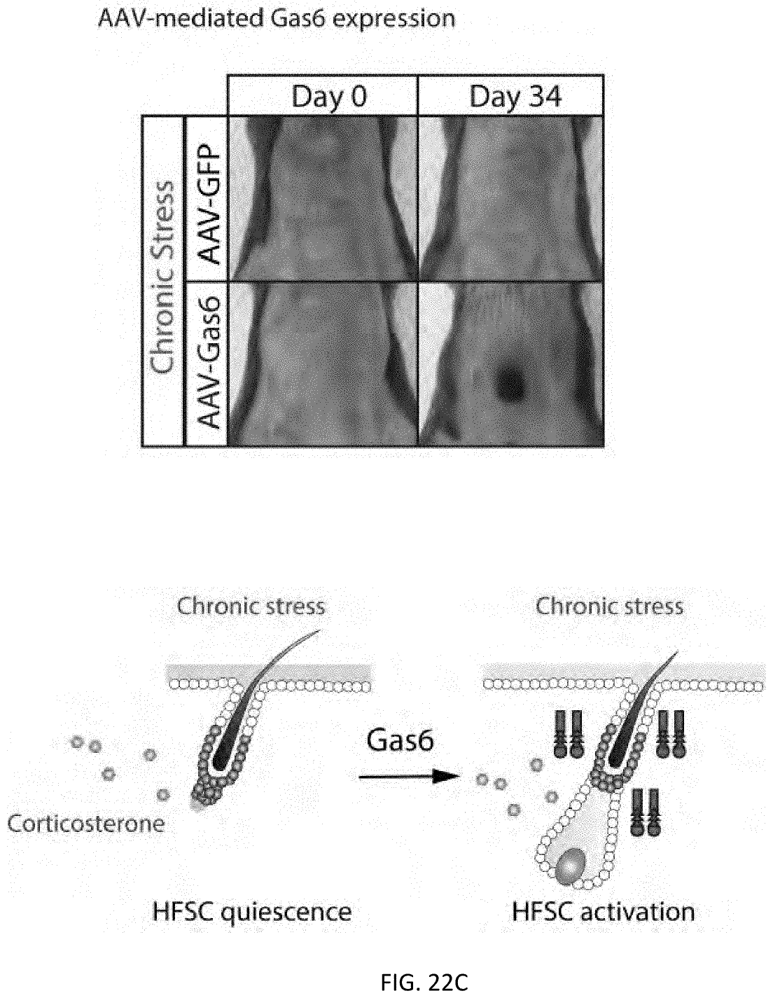

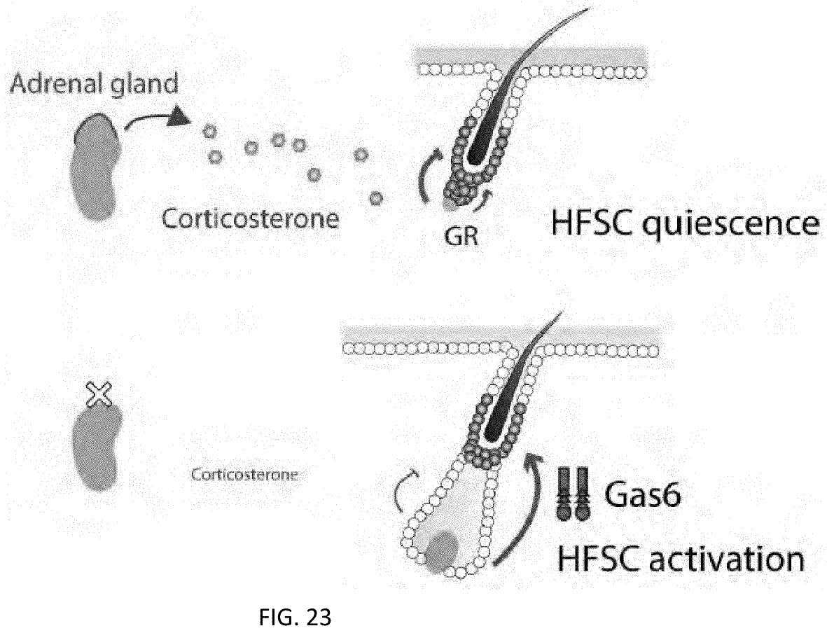

The findings thus far suggest that adrenal gland-derived corticosterone inhibits Gas6 expression in the dermal niche to enforce telogen under normal physiological conditions. Since elevation of corticosterone is responsible for extending telogen in stressed mice, it was asked whether the mechanisms identified here might be harnessed to counteract the effects of corticosterone elevation and to promote HFSC activation under stress. To this end, mice were examined subjected to chronic unpredictable stress, asking if extended telogen would be shortened if GR was depleted from the dermal niche but not elsewhere (FIG. 13A). Indeed, it was found that Pdgfra-CreER; GRfl/fl mice displayed significantly shorter telogen compared to control mice in the chronic unpredictable stress model (FIG. 13B). These data suggest that depletion of GR from the dermal niche is sufficient to rescue the delayed hair cycle entry upon stress. Then qRT-PCR was used to examine Gas6 expression levels in DP isolated from stressed mice, and found that Gas6 levels were indeed significantly down-regulated in DP upon stress (FIG. 5D).

To determine if restoring Gas6 expression would be sufficient to overcome stress-induced effects on HFSCs, AAV-CAG-Gas6 was injected intradermally, and subjected the mice to chronic unpredictable stress or long-term corticosterone feeding (FIG. 5E). It was found that overexpression of Gas6 effectively mitigated prolonged telogen caused by either condition (FIGS. 5F-5G), suggesting that restoration of Gas6 expression was sufficient to promote HFSC activation in a high corticosterone environment. Altogether, the data suggest that corticosterone controls HFSC activity by inhibiting the expression of Gas6 in the DP. When corticosterone levels drop, elevated Gas6 expression promotes HFSC activation and hair follicle regeneration. Conversely, when corticosterone levels are elevated, Gas6 expression is inhibited, HFSCs remain quiescent, and hair follicle regeneration is blocked (FIG. 5H).

### Corticosterone Regulates HFSCs via the Dermal Niche

Stem cells respond to, and integrate, both local and systemic inputs to couple tissue regeneration with the animal's overall physiological state⁴⁷⁻⁵⁰. Proliferation of Drosophila germline stem cells is under direct control of insulin and the steroid hormone twenty-hydroxyecdysone (20E)^{51,52}. In mammals, haematopoietic stem cell maintenance is influenced by oestrogen and liver-derived thrombopoietin^{53,54}. These examples demonstrate how systemic hormones can act on stem cells directly to alter their behaviours. Here, a physiological mechanism is unravelled in which a systemic factor regulates a mammalian stem cell by inhibiting a niche factor.

Exposure to acute sound stress causes apoptosis of the hair bulb through the neuropeptide substance P55. Here, a distinct mechanism is identified by which chronic stress elevates corticosterone levels, blocking the ability of HFSCs to enter anagen and to regenerate new hair follicles. Stress is a major risk factor for telogen effluvium, a common hair loss condition characterized by large numbers of hair follicles in telogen at the same time⁵⁶. It is conceivable that stress might accelerate progression of other hair loss conditions. The study identifies a mechanism through which stress can inhibit tissue regeneration, and pinpoints a pathway that might be targeted therapeutically to overcome this stress-induced regeneration block.

### Gas6-AXL Signalling: a Potent Pathway for HFSC Activation

DP is a key niche cell type that tunes the ability of HFSCs to transition from quiescence to activation^{14,38,57-59}. Here it is shown that Gas6 expression in DP is kept at a modest level by circulating corticosterone, providing an interesting example in which an activating niche factor is under constant suppression by a systemic regulator. The corticosterone-Gas6 axis might function to override other niche factors, extending or shortening telogen based on the overall physiological state of the organism. Since corticosterone levels display a diurnal rhythm, and are subject to seasonal changes^{60,61}, it is also possible that the corticosterone-Gas6 axis helps fine-tune activity of HFSCs based on circadian rhythm⁶²⁻⁶⁴, or contributes to regulating seasonal moulting in wild animals.

Gas6-AXL signalling is best known for its role in tumor progression and innate immunity⁶⁵. ZIKA viruses have also been shown to enter human cells via the AXL receptor^{66,67}. Here, a previously unknown function of Gas6-AXL signalling was identified in promoting HFSC activation and hair follicle growth. Moreover, it is shown that supplying Gas6 locally is sufficient to overcome the arrested regeneration imposed by elevated corticosterone levels in both control and stressed situations. It will be interesting to investigate the therapeutic potential of the Gas6-AXL pathway in promoting hair growth.

Overriding Stem Cell Quiescence without Exhaustion

Quiescence has been postulated to be crucial to preserve the ability of stem cells to regenerate tissues long-term, particularly for somatic stem cells that cycle rarely^{3,19,23,25,68,69}. Here, a pathway was identified through which HFSCs can be activated continuously without exhaustion. This finding suggests that at least for HFSCs, quiescence and telogen are both dispensable for tissue regeneration long-term. The results also suggest that without corticosterone, the regenerative capacity of HFSCs does not decline much with age, as it was found that old ADX animals regenerated hair follicles at a frequency similar to very young control animals.

Stem cell quiescence is known to prevent tumor initiation by HFSCs carrying active oncogenes or inactive tumor suppressors⁷⁰. This said, apparent signs of hyperplasia in the ADX mice or Gas6 overexpression mice were not observed, suggesting that, in the absence of tumor-associated mutations, loss of HFSC quiescence due to modulations of the corticosterone-Gas6 axis does not automatically lead to aberrant overgrowth.

In conclusion, the study showcases an unprecedented regenerative capacity of mouse HFSCs when released from the systemic control of corticosterone. The findings also open the door to future investigations into corticosterone-mediated regulation of stem cell quiescence in other systems, as well as potential therapeutic strategies to combat the detrimental impact of stress.

### REFERENCES

1. Cheung, T. H. & Rando, T. A. Molecular regulation of stem cell quiescence. Nat Rev Mol Cell Biol 14, 329-340 (2013).

2. Tumbar, T. et al. Defining the epithelial stem cell niche in skin. Science 303, 359-363 (2004).

3. Yi, R. Concise Review: Mechanisms of Quiescent Hair Follicle Stem Cell Regulation. Stem Cells 35, 2323-2330 (2017).

4. Cheng, T. et al. Hematopoietic stem cell quiescence maintained by p21cip1/waf1. Science 287, 1804-1808 (2000).

5. Cho, I. J. et al. Mechanisms, Hallmarks, and Implications of Stem Cell Quiescence. Stem Cell Reports 12, 1190-1200 (2019).

6. Pinho, S. & Frenette, P. S. Haematopoietic stem cell activity and interactions with the niche. Nat Rev Mol Cell Biol 20, 303-320 (2019).

7. Gross, K. L. & Cidlowski, J. A. Tissue-specific glucocorticoid action: a family affair. Trends Endocrinol Metab 19, 331-339 (2008).

8. Chrousos, G. P. Stress and disorders of the stress system. Nat Rev Endocrinol 5, 374-381 (2009).

9. Pierce, H. et al. Cholinergic Signals from the CNS Regulate G-CSF-Mediated HSC Mobilization from Bone Marrow via a Glucocorticoid Signaling Relay. Cell Stem Cell 20, 648-658 e644 (2017).

10. Chetty, S. et al. Stress and glucocorticoids promote oligodendrogenesis in the adult hippocampus. Mol Psychiatry 19, 1275-1283 (2014).

11. Besnard, A. et al. Targeting Kruppel-like Factor 9 in Excitatory Neurons Protects against Chronic Stress-Induced Impairments in Dendritic Spines and Fear Responses. Cell Rep 23, 3183-3196 (2018).

12. Muller-Rover, S. et al. A comprehensive guide for the accurate classification of murine hair follicles in distinct hair cycle stages. J Invest Dermatol 117, 3-15 (2001).

13. Hsu, Y. C., Li, L. & Fuchs, E. Transit-amplifying cells orchestrate stem cell activity and tissue regeneration. Cell 157, 935-949 (2014).

14. Greco, V. et al. A two-step mechanism for stem cell activation during hair regeneration. Cell Stem Cell 4, 155-169 (2009).

15. Morris, R. J. et al. Capturing and profiling adult hair follicle stem cells. Nat Biotechnol 22, 411-417 (2004).

16. Rompolas, P., Mesa, K. R. & Greco, V. Spatial organization within a niche as a determinant of stem-cell fate. Nature 502, 513-518 (2013).

17. Chen, C. C. et al. Regenerative hair waves in aging mice and extra-follicular modulators follistatin, dkk1, and sfrp4. J Invest Dermatol 134, 2086-2096 (2014).

18. Keyes, B. E. et al. Nfatcl orchestrates aging in hair follicle stem cells. Proc Natl Acad Sci USA 110, E4950-4959 (2013).

19. Lay, K., Kume, T. & Fuchs, E. FOXC1 maintains the hair follicle stem cell niche and governs stem cell quiescence to preserve long-term tissue-regenerating potential. Proc Natl Acad Sci USA 113, E1506-1515 (2016).

20. Hoi, C. S. et al. Runxl directly promotes proliferation of hair follicle stem cells and epithelial tumor formation in mouse skin. Mol Cell Biol 30, 2518-2536 (2010).

21. Castellana, D., Paus, R. & Perez-Moreno, M. Macrophages contribute to the cyclic activation of adult hair follicle stem cells. PLoS Biol 12, e1002002 (2014).

22. Flores, A. et al. Lactate dehydrogenase activity drives hair follicle stem cell activation. Nat Cell Biol 19, 1017-1026 (2017).

23. Wang, L., Siegenthaler, J. A., Dowell, R. D. & Yi, R. Foxcl reinforces quiescence in self-renewing hair follicle stem cells. Science 351, 613-617 (2016).

24. Wang, E. C. E., Dai, Z., Ferrante, A. W., Drake, C. G. & Christiano, A. M. A Subset of TREM2(+) Dermal Macrophages Secretes Oncostatin M to Maintain Hair Follicle Stem Cell Quiescence and Inhibit Hair Growth. Cell Stem Cell 24, 654-669 e656 (2019).

25. Horsley, V., Aliprantis, A. O., Polak, L., Glimcher, L. H. & Fuchs, E. NFATc1 balances quiescence and proliferation of skin stem cells. Cell 132, 299-310 (2008).

26. Plikus, M. V. et al. Cyclic dermal BMP signalling regulates stem cell activation during hair regeneration. Nature 451, 340-344 (2008).

27. Craven, A. J. et al. Prolactin delays hair regrowth in mice. J Endocrinol 191, 415-425 (2006).

28. Goldstein, J. et al. Calcineurin/Nfatc1 signaling links skin stem cell quiescence to hormonal signaling during pregnancy and lactation. Genes Dev 28, 983-994 (2014).

29. Rose, J. & Sterner, M. The role of the adrenal glands in regulating onset of winter fur growth in mink (Mustela vison). J Exp Zool 262, 469-473 (1992).

30. Butcher, E. O. Hair growth in adrenalectomized, and, adrenalectomized thyroxin-treated rats. Am J Physiol 120, 427-434 (1937).

31. Hsu, Y. C., Pasolli, H. A. & Fuchs, E. Dynamics between stem cells, niche, and progeny in the hair follicle. Cell 144, 92-105 (2011).

32. Matsumura, H. et al. Hair follicle aging is driven by transepidermal elimination of stem cells via COL17A1 proteolysis. Science 351, aad4395 (2016).

33. Walczak, E. M. & Hammer, G. D. Regulation of the adrenocortical stem cell niche: implications for disease. Nat Rev Endocrinol 11, 14-28 (2015).

34. Ulrich-Lai, Y. M. & Herman, J. P. Neural regulation of endocrine and autonomic stress responses. Nat Rev Neurosci 10, 397-409 (2009).

35. Tye, K. M. et al. Dopamine neurons modulate neural encoding and expression of depression-related behaviour. Nature 493, 537-541 (2013).

36. Heidt, T. et al. Chronic variable stress activates hematopoietic stem cells. Nat Med 20, 754-758 (2014).

37. Weikum, E. R., Knuesel, M. T., Ortlund, E. A. & Yamamoto, K. R. Glucocorticoid receptor control of transcription: precision and plasticity via allostery. Nat Rev Mol Cell Biol 18, 159-174 (2017).

38. Enshell-Seijffers, D., Lindon, C., Kashiwagi, M. & Morgan, B. A. beta-catenin activity in the dermal papilla regulates morphogenesis and regeneration of hair. Dev Cell 18, 633-642 (2010).

39. Rompolas, P. et al. Live imaging of stem cell and progeny behaviour in physiological hair-follicle regeneration. Nature 487, 496-499 (2012).

40. Festa, E. et al. Adipocyte lineage cells contribute to the skin stem cell niche to drive hair cycling. Cell 146, 761-771 (2011).

41. Zhang, B. et al. Hair follicles' transit-amplifying cells govern concurrent dermal adipocyte production through Sonic Hedgehog. Genes Dev 30, 2325-2338 (2016).

42. Clavel, C. et al. Sox2 in the dermal papilla niche controls hair growth by fine-tuning BMP signaling in differentiating hair shaft progenitors. Dev Cell 23, 981-994 (2012).

43. Stitt, T. N. et al. The anticoagulation factor protein S and its relative, Gas6, are ligands for the Tyro 3/Axl family of receptor tyrosine kinases. Cell 80, 661-670 (1995).

44. Rothlin, C. V., Carrera-Silva, E. A., Bosurgi, L. & Ghosh, S. TAM receptor signaling in immune homeostasis. Annu Rev Immunol 33, 355-391 (2015).

45. Holland, S. J. et al. R428, a selective small molecule inhibitor of Axl kinase, blocks tumor spread and prolongs survival in models of metastatic breast cancer. Cancer Res 70, 1544-1554 (2010).

46. Goldstein, J. M. et al. In Situ Modification of Tissue Stem and Progenitor Cell Genomes. Cell Rep 27, 1254-1264 e1257 (2019).

47. Jasper, H. & Jones, D. L. Metabolic regulation of stem cell behavior and implications for aging. Cell Metab 12, 561-565 (2010).

48. O'Brien, L. E. & Bilder, D. Beyond the niche: tissue-level coordination of stem cell dynamics. Annu Rev Cell Dev Biol 29, 107-136 (2013).

49. Frenette, P. S., Pinho, S., Lucas, D. & Scheiermann, C. Mesenchymal stem cell: keystone of the hematopoietic stem cell niche and a stepping-stone for regenerative medicine. Annu Rev Immunol 31, 285-316 (2013).

50. Morrison, S. J. & Spradling, A. C. Stem cells and niches: mechanisms that promote stem cell maintenance throughout life. Cell 132, 598-611 (2008).

51. Ables, E. T. & Drummond-Barbosa, D. The steroid hormone ecdysone functions with intrinsic chromatin remodeling factors to control female germline stem cells in Drosophila. Cell Stem Cell 7, 581-592 (2010).

52. LaFever, L. & Drummond-Barbosa, D. Direct control of germline stem cell division and cyst growth by neural insulin in Drosophila. Science 309, 1071-1073 (2005).

53. Nakada, D. et al. Oestrogen increases haematopoietic stem-cell self-renewal in females and during pregnancy. Nature 505, 555-558 (2014).

54. Decker, M., Leslie, J., Liu, Q. & Ding, L. Hepatic thrombopoietin is required for bone marrow hematopoietic stem cell maintenance. Science 360, 106-110 (2018).

55. Arck, P. C. et al. Stress inhibits hair growth in mice by induction of premature catagen development and deleterious perifollicular inflammatory events via neuropeptide substance P-dependent pathways. Am J Pathol 162, 803-814 (2003).

56. Rebora, A. Proposing a Simpler Classification of Telogen Effluvium. Skin Appendage Disord 2, 35-38 (2016).

57. Rendl, M., Polak, L. & Fuchs, E. BMP signaling in dermal papilla cells is required for their hair follicle-inductive properties. Genes Dev 22, 543-557 (2008).

58. Oshimori, N., Oristian, D. & Fuchs, E. TGF-beta promotes heterogeneity and drug resistance in squamous cell carcinoma. Cell 160, 963-976 (2015).

59. Hawkshaw, N. J. et al. Identifying novel strategies for treating human hair loss disorders: Cyclosporine A suppresses the Wnt inhibitor, SFRP1, in the dermal papilla of human scalp hair follicles. PLoS Biol 16, e2003705 (2018).

60. Cahill, S., Tuplin, E. & Holahan, M. R. Circannual changes in stress and feeding hormones and their effect on food-seeking behaviors. Front Neurosci 7, 140 (2013).

61. Nader, N., Chrousos, G. P. & Kino, T. Interactions of the circadian CLOCK system and the HPA axis. Trends Endocrinol Metab 21, 277-286 (2010).

62. Janich, P. et al. The circadian molecular clock creates epidermal stem cell heterogeneity. Nature 480, 209-214 (2011).

63. Lin, K. K. et al. Circadian clock genes contribute to the regulation of hair follicle cycling. PLoS Genet 5, e1000573 (2009).

64. Plikus, M. V. et al. Local circadian clock gates cell cycle progression of transient amplifying cells during regenerative hair cycling. Proc Natl Acad Sci USA 110, E2106-2115 (2013).

65. Lemke, G. & Rothlin, C. V. Immunobiology of the TAM receptors. Nat Rev Immunol 8, 327-336 (2008).

66. Nowakowski, T. J. et al. Expression Analysis Highlights AXL as a Candidate Zika Virus Entry Receptor in Neural Stem Cells. Cell Stem Cell 18, 591-596 (2016).

67. Meertens, L. et al. Axl Mediates ZIKA Virus Entry in Human Glial Cells and Modulates Innate Immune Responses. Cell Rep 18, 324-333 (2017).

68. Nakamura-Ishizu, A., Takizawa, H. & Suda, T. The analysis, roles and regulation of quiescence in hematopoietic stem cells. Development 141, 4656-4666 (2014).

69. Kippin, T. E., Martens, D. J. & van der Kooy, D. p21 loss compromises the relative quiescence of forebrain stem cell proliferation leading to exhaustion of their proliferation capacity. Genes Dev 19, 756-767 (2005).

70. White, A. C. et al. Stem cell quiescence acts as a tumour suppressor in squamous tumours. Nat Cell Biol 16, 99-107 (2014).

71. Mittelstadt, P. R., Monteiro, J. P. & Ashwell, J. D. Thymocyte responsiveness to endogenous glucocorticoids is required for immunological fitness. The Journal of clinical investigation 122, 2384-2394 (2012).

72. Kang, S. H., Fukaya, M., Yang, J. K., Rothstein, J. D. & Bergles, D. E. NG2+ CNS glial progenitors remain committed to the oligodendrocyte lineage in postnatal life and following neurodegeneration. Neuron 68, 668-681 (2010).

73. Arnold, K. et al. Sox2(+) adult stem and progenitor cells are important for tissue regeneration and survival of mice. Cell stem cell 9, 317-329 (2011).

74. Srinivas, S. et al. Cre reporter strains produced by targeted insertion of EYFP and ECFP into the ROSA26 locus. BMC Dev Biol 1, 4 (2001).

75. Plikus, M. V. & Chuong, C. M. Complex hair cycle domain patterns and regenerative hair waves in living rodents. J Invest Dermatol 128, 1071-1080 (2008).

76. Patro, R., Duggal, G., Love, M. I., Irizarry, R. A. & Kingsford, C. Salmon provides fast and bias-aware quantification of transcript expression. Nat Methods 14, 417-419 (2017).

77. Love, M. I., Huber, W. & Anders, S. Moderated estimation of fold change and dispersion for RNA-seq data with DESeq2. Genome Biol 15, 550 (2014).

78. Mou, H. et al. Dual SMAD Signaling Inhibition Enables Long-Term Expansion of Diverse Epithelial Basal Cells. Cell stem cell 19, 217-231 (2016).

79. Nowak, J. A. & Fuchs, E. Isolation and culture of epithelial stem cells. Methods in molecular biology 482, 215-232 (2009).

What is claimed is:

1. A method of modulating hair growth in an individual in need thereof comprising administering to the individual an agent that modulates a Gas6-Tyro3/Axl/Mertk (TAM) interaction or pathway.

2. A method of modulating hair follicle stem cell (HFSC) activation in an individual in need thereof comprising administering to the individual an agent that modulates a Gas6-Tyro3/Axl/Mertk (TAM) interaction or pathway.

3. The method of claim 1 or 2, wherein the TAM interaction or pathway is an AXL interaction or pathway.

4. The method of claim 1 or 2, wherein the TAM interaction or pathway is a Tyro3 interaction or pathway.

5. The method of claim 1 or 2, wherein the TAM interaction or pathway is a Mertk interaction or pathway.

6. The method of claim 1 or 2, wherein the agent modulates Gas6 activity or expression.

7. The method of claim 6, wherein the agent increases Gas6 activity or expression.

8. The method of claim 6, wherein the agent decreases Gas6 activity or expression.

9. The method of claim 1, wherein the agent increases hair growth.

10. The method of claim 1, wherein the agent decreases hair growth.

11. The method of claim 1 or 2, wherein the agent is administered using an AAV vector.

12. The method of claim 11, wherein the AAV is AAV8.

13. The method of claim 11, wherein the agent is administered through intradermal injection.

14. The method of claim 1 or 2, wherein the agent modulates AXL activity or expression.

15. The method of claim 14, wherein the agent increases AXL activity or expression.

16. The method of claim 14, wherein the agent decreases AXL activity or expression.

17. The method of claim 1, wherein the method increases hair growth.

18. The method of claim 17, wherein the method increases hair growth by at least 25% relative to a suitable control.

19. The method of claim 1, wherein the method decreases hair growth.

20. The method of claim 19, wherein the method decreases hair growth by at least 25% relative to a suitable control.

21. The method of claim 1, wherein the method increases hair growth under stress conditions.

22. The method of claim 2, wherein the method increases HFSC activation under stress conditions.

23. The method of claim 21 or 22, wherein the stress condition is evidenced by elevated corticosterone or hair loss.

24. The method of claim 23, wherein the hair loss condition is telogen effluvium.

25. The method of claim 2, wherein the agent increases HFSC activation.

26. The method of claim 2, wherein the agent decreases HFSC activation.

27. The method of claim 2, wherein the method increases HFSC activation.

28. The method of claim 27, wherein the method increases HFSC activation by at least 25% relative to a suitable control.

29. The method of claim 2, wherein the method decreases HFSC activation.

30. The method of claim 29, wherein the method decreases HFSC activation by at least 25% relative to a suitable control.
